(19) United States
(12) Patent Application Publication
(10) Pub. No.: US 2021/0343515 A1
(43) Pub. Date: Nov. 4, 2021

(54) SYSTEMS AND METHODS FOR MICROARRAY DROPLET IONIZATION ANALYSIS

(71) Applicant: BOARD OF REGENTS, THE UNIVERSITY OF TEXAS SYSTEM, Austin, TX (US)

(72) Inventors: Livia Schiavinato EBERLIN, Austin, TX (US); Marta Sans ESCOFET, Austin, TX (US); Bryan R. WYGANT, Austin, TX (US); Jialing ZHANG, Austin, TX (US); C. Buddie MULLINS, Austin, TX (US)

(21) Appl. No.: 16/620,298

(22) PCT Filed: Jun. 8, 2018
(86) PCT No.: PCT/US2018/036648

§ 371 Date: Dec. 6, 2019

Related U.S. Application Data
(60) Provisional application No. 62/517,050, filed on Jun. 8, 2017.

(51) Int. Cl. H01J 49/00; H01J 49/04; G01N 33/68

(52) U.S. Cl. CPC H01J 49/0004; G01N 33/6848; H01J 49/0431; H01J 49/0477

(57) ABSTRACT

Method and devices are provided for imaging a surface, such as a biological tissue sample, by mass spectrometry. In certain aspects, devices of the embodiments allow for the placement and collection of a plurality of spatially separated liquid droplets on a sample and delivery of the droplets with extracted sample analytes for mass spectrometry analysis.

This application claims the benefit of U.S. Provisional Patent Application No. 62/517,050, filed Jun. 8, 2017, the entirety of which is incorporated herein by reference.

The invention was made with government support under Grant No. CA190783 awarded by the National Institutes of Health. The government has certain rights in the invention.

### BACKGROUND OF THE INVENTION

### 1. Field of the Invention

The present invention relates generally to the field of mass spectrometry imaging. More particularly, it concerns ambient mass spectrometry imaging technologies.

### 2. Description of Related Art

Mass spectrometry imaging (MSI) is a powerful method for the analysis of biological tissue as it can provide direct investigation of the spatial distribution and chemical identification of hundreds of analyte molecules with high specificity and sensitivity (McDonnell and Heeren, 2007). Abnormalities in metabolite, lipid and protein levels are known to occur in a variety of diseases, and can be elucidated by molecular imaging (Eberlin et al., 2014; Eberlin et al., 2012; Guenther et al., 2015; Zhang et al., 2016). Nevertheless, the molecular complexity and spatial heterogeneities of biological tissue involved in human disorders calls for new MSI technologies. These technologies should aim to provide comprehensive and sensitive analysis of molecular species with fine spatial control, in order to improve disease diagnostics and provide a better understanding about disease states (Giesen et al., 2014).

Matrix assisted laser desorption ionization (MALDI) is the most widely used imaging technology for molecular analysis of tissue samples. Protein and peptides have been extensively characterized by MALDI imaging with high spatial resolution (25-250 μm) (Seeley and Caprioli, 2011). However, the requirements of targeted matrix deposition, high vacuum conditions and chemical noise have prevented its broad application for high-throughput analysis of biological samples. The development of ambient ionization in 2006 revolutionized the field of MSI, allowing biological samples to be analyzed in situ at atmospheric pressure (Cooks et al., 2006). The first and most employed ambient ionization technique is desorption electrospray ionization (DESI), which utilizes a solvent electrospray to desorb molecular species present on the sample surface (Venter et al., 2006). The capabilities of DESI have been explored for multiple clinical applications, such as developing molecular models for rapid and accurate cancer diagnosis (Ifa and Eberlin, 2016). However, molecular sampling at ambient conditions is also associated with the following challenges: (1) poor sensitivity due to inefficient droplet transmission at atmospheric pressure, (2) limited range of molecular detection in complex samples based on limitations from desorption mechanisms (max. m/z 3,000), (3) lack of spatial control and high resolution compared to laser desorption technologies (150-250 μm), and (4) matrix effects from molecular interferences in complex systems due to lack of chromatographic separation (Harris et al., 2011).

MSI has emerged as an exceptional technology for molecular and spatial evaluation of biological samples. In particular, ambient ionization MSI techniques, powered by the development of desorption electrospray ionization (DESI) in 2004, have allowed the direct analysis of tissue samples, with minimal pretreatment, providing powerful capabilities suitable for clinical applications. However, various challenges are associated with molecular sampling in the open environment, preventing the widespread of ambient MSI technologies.

### SUMMARY OF THE INVENTION

Exemplary embodiments of the present disclosure include a new ambient MSI technique, MicroArray Droplet Ionization (MADI), which provides enhanced sensitivity, improved spatial control/resolution and comprehensive molecular imaging. In certain embodiments, MADI combines a piezoelectric picoliter dispenser, to form an array of microdroplets onto the sample surface with controlled spatial resolution, and a conductive emitter to aspirate/ionize the microdroplets for sensitive molecular detection. Specific embodiments demonstrate the capabilities of MADI-MS by imaging mouse brain, human brain, and human ovarian tissue samples at different spatial resolutions. MADI-MS can also be applied towards the sensitive and comprehensive profiling of human ovarian cell lines in particular embodiments. Test results demonstrate the capabilities and advantages of MADI-MS for sensitive biological sample imaging and analysis.

Certain embodiments of the present disclosure provide an apparatus for producing samples for mass spectrometry analysis. In one embodiment, the apparatus for producing samples for mass spectrometry analysis comprises a solvent dispenser configured to dispense droplets of solvent on a sample comprising an analyte, a conduit configured to transfer the droplets of solvent and the analyte from the sample to a mass spectrometer and configured to provide an electrical potential (e.g., for ionization), and, optionally, a heat conduit configured to heat (and further ionize) the droplets of solvent and the analyte prior to transfer to the mass spectrometer.

In some aspects, the solvent dispenser comprises a piezoelectric actuator. In certain aspects, the droplets of solvent are between 5 and 50 picoliters, such as between 10 and 30 picoliters. In particular aspects, the droplets of solvent are approximately 22 picoliters.

In certain aspects, the solvent dispenser is configured to dispense droplets of solvent in a grid pattern. In some aspects, the droplets of solvent are spaced apart between 0.05 mm and 1.0 mm in the grid pattern (e.g., 0 between 0.05, 0.1, 0.2, 0.3, 0.4 0.5, 0.6, 0.7, 0.8, 0.9 and 1.0 or any range derivable therein).

In additional aspects, the apparatus further comprises a sample retainer configured to retain the sample. In some aspects, the apparatus further comprises an actuator configured to move the sample retainer in two orthogonal directions. In certain aspects, the apparatus further comprises an actuator configured to move the sample retainer in three orthogonal directions.

In some aspects, the apparatus further comprises a heated conduit that comprises a heating element and a voltage source. In certain aspects, the heating element is configured to be heated to a temperature between 250 and 350 Celsius (e.g., 250, 260, 270, 280, 290, 300, 310, 320, 330, 340, or 350 Celsius). In particular aspects, the heating element is configured to be heated to a temperature of approximately 300 Celsius.

In certain aspects, the conduit is a capillary tube comprising a first end proximal to the solvent dispenser and a second end distal from the solvent dispenser. In specific aspects, the capillary tube comprises an electrically conductive material. In one particular aspect, the electrically conductive material is a metal coating proximal to the first end of the capillary tube. In some aspects, the metal coating is platinum.

In some aspects, the ionization device is configured to apply a voltage differential between the metal coating and the second end of the capillary tube. In certain aspects, the capillary tube comprises an outer diameter between 300 and 400 micrometers (μm) (e.g., 300, 310, 320, 330, 340, 350, 360, 370, 380, 390 or 400 μm) and inner diameter between 50 and 150 micrometers (μm) (e.g., 50, 60, 70, 80, 90, 100, 110, 120, 130, 140, or 150 μm). In particular aspects, the capillary tube comprises an outer diameter of approximately 360 micrometers (μm) and inner diameter of approximately 100 micrometers (μm). In some aspects, the capillary tube is a silica tube.

In additional aspects, the apparatus further comprises a mass spectrometer coupled to the conduit.

In some aspects, the solvent dispenser comprises a plurality of pneumatic lines, a plurality of reservoirs, and a plurality of dispensing tips, where the plurality of pneumatic lines are configured to transport solvent out of the plurality of reservoirs and into dispensing tips.

In another embodiment, the present disclosure provides a method for imaging a surface comprising applying a discrete volume of a solvent to a plurality of distinct sites on the surface, the discrete volume of solvent being applied through a dispenser, individually collecting and ionizing the discrete volumes of applied solvent to obtain a plurality of ionized liquid samples, wherein the collecting is through a solvent conduit, and individually subjecting the plurality of ionized liquid samples to mass spectrometry analysis. In particular aspects, the method is performed using an apparatus of the embodiments.

In some aspects, the plurality of distinct sites are spaced essentially uniformly from one another across the surface. In certain aspects, the plurality of distinct sites are arranged in a grid patter over the surface. In some aspects, the plurality of distinct sites comprise at least 10 sites. In particular aspects, the plurality of distinct sites comprise 100 to 5,000 sites, such as 200, 500, 1,000, 2,000, 3,000, 4,000, or 5,000 sites. In certain aspects, the location of each of the plurality of distinct sites is recorded and correlated to the mass spectrometry analysis obtained for the liquid sample corresponding to the site. In some aspects, the plurality of distinct sites are separated by about 0.05 to 1.0 mm (e.g., between 0.05, 0.1, 0.2, 0.3, 0.4 0.5, 0.6, 0.7, 0.8, 0.9 and 1.0 or any range derivable therein).

In further aspects, the method further comprises producing an array of data from the mass spectrometry analysis of the plurality of sites to image the surface.

In additional aspects, the method is automated. In some aspects, the steps of applying a discrete volume of a solvent to a plurality of distinct sites on the surface, the discrete volume of solvent being applied through a dispenser, individually collecting and ionizing the discrete volumes of applied solvent to obtain a plurality of ionized liquid samples are performed by a robot.

In some aspects, the discrete volume of a solvent is not applied as a spray. In certain aspects, the discrete volume of a solvent is applied as a droplet. In some aspects, the discrete volume of a solvent is between 5 and 50 or 10 and 30 picoliters. In some aspects, the discrete volume of a solvent is applied at using a pressure of less than 100 psig. In particular aspects, the discrete volume of a solvent is applied at using a pressure of less than 10 psig.

In certain aspects, individually collecting and ionizing the discrete volumes comprises applying an electrical potential and/or heat to the collected solvent. In some aspects, applying heat comprises heating to a temperature between 250 and 350 Celsius (e.g., 250, 260, 270, 280, 290, 300, 310, 320, 330, 340, or 350 Celsius). In particular aspects, the electrical potential comprises at least 0.5 kV, such as between about 1.0 and 5.0 kV or between about 1.0 and 2.0 kV.

In some aspects, the discrete volume of a solvent is applied using a mechanical pump to move the solvent through the dispenser. In other aspects, the discrete volume of a solvent is applied using a piezoelectric actuator to move the solvent through the dispenser. In some aspects, the solvent conduit is a capillary tube. In particular aspects, the solvent conduit is composed of silica. In some aspects, the solvent conduit has an inner diameter between 50 and 150 micrometers (μm) (e.g., 50, 60, 70, 80, 90, 100, 110, 120, 130, 140, or 150 μm). In certain aspects, the solvent conduit comprises an electrically conductive material. In specific aspects, the electrically conductive material is a metal coating, such as platinum.

In some aspects, the solvent is applied through a dispenser that is separate from the collection conduit. In specific aspects, the solvent comprises methanol, chloroform, formic acid, dimethylformamide (DMF) or acetonitrile (ACN). In certain aspects, the solvent comprises a mixture of DMF and ACN. In some aspects, the solvent is essentially free of water. In certain aspects, the solvent comprises an agent that increases surface tension. In particular aspects, the solvent comprises a surfactant or a supercharging reagent.

In certain aspects, collecting the applied solvent is between 0.05 and 10 seconds after the applying step. In some aspects, the surface comprises a biological material. In particular aspects, the biological material is a tissue section. In specific aspects, the biological material is resected tissue from a subject. In one aspects, the resected tissue is a tumor.

In some aspects, the mass spectrometry comprises ambient ionization MS.

In certain aspects the dispenser comprises a plurality of pneumatic lines, a plurality of reservoirs, and a plurality of dispensing tips; and applying the discrete volume of solvent through the dispenser comprises transporting the solvent from plurality of reservoirs, through the plurality of pneumatic lines and into dispensing tips.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B: Lipid ionization enhancement with high voltage. A) Representative lipid profiles at 1 and 0 kV applied to conductive tip. B) Total ion chromatogram for 4 lipid droplets at 1 kV.

FIG. 2: Arm holder and 2D moving stage mounted at the front end of the mass spectrometer (MS).

FIGS. 3A-3B: A) Schematic for aspiration of lipid droplets deposited on a PTFE surface. B) Total ion chromatogram.

FIGS. 4A-4C: A) Grey and white matter on a stained rat brain tissue section B) Representative profile from grey matter by extraction of droplet 1 (top) and by DESI-MS (bottom) C) Representative profile from grey matter by extraction of droplet 2 (top) and by DESI-MS (bottom).

FIGS. 5A-5B: A) 1 nL droplet array for 6 droplet spots on a rat brain tissue sample prior to analysis, achieving a spatial resolution of ˜500 μm. B) Total ion chromatogram corresponding to the aspiration of 6 droplets.

FIGS. 6A-6B: A) Representative ion images for high-grade SC tissue by DESI-MSI, with the same tissue slide stained by H & E. B) Microscope images for the same tissue sample in A showing the tumor heterogeneities at different magnifications.

FIGS. 7A-7C: A) Schematic depicting droplet array based mass spectrometry. B) Schematic depicting solvent droplets sequentially dispensed and extracted/ionized with precise time control. C) Schematic depicting dispenser with pneumatic lines and reservoirs.

FIG. 8: High spatial control is achieved by tuning the volume deposited by the picoliter dispenser onto the tissue sample. As the volume deposited decreases, the spatial resolution decreases accordingly following a logarithmic trend.

FIG. 9: Schematic of the MADI setup designed and developed for the transport and ionization of solvated analyte droplets from tissue samples.

FIG. 10: Photograph of array of DMF droplets deposited onto a mouse brain sample.

FIG. 11: Photograph of MADI setup coupled to a Q Exactive Orbitrap system.

FIG. 12: Photograph of silica emitter aligned with the transfer tube connected to the mas spectrometer (MS) inlet.

FIG. 13: Graphs showing optimization of the voltage applied to the capillary emitter [Panels (a,b)] and temperature provided to the inlet of the MS system [Panels (c,d)]. Effect on MADI performance was evaluated based on total ion current (a,c) and absolute abundance at certain m/z values grouped according to molecular class: metabolites, fatty acids, and lipids [Panels (b,d)].

FIG. 14: Panel (a) shows MADI-MS ion images obtained at different spatial resolutions from serial mouse brain tissue sections. Representative MADI-MS spectra and comparisons to DESI-MS spectra at the same spatial resolution from grey matter are shown in Panel (b) and for white matter regions are shown in Panel (c).

FIG. 15 Panel (a) shows MADI-MS imaging of ovarian carcinoma samples. Panel (b) shows representative MADI-MS spectra from high-grade serous carcinoma (top), low-grade serous carcinoma (middle), and normal ovarian tissue (bottom). Lipid species are color-coded according to lipid class.

FIG. 16 Panel (a) shows MADI-MS imaging of a glioblastoma tumor sample (top) and normal brain tissue (bottom). Panel (b) shows representative MADI-MS spectra from glioblastoma tissue (top), grey matter (middle) and white matter (bottom) normal brain tissue. Lipid species are color-coded according to lipid class.

FIG. 17 shows representative MADI-MS profiles obtained from the analysis of human ovarian tumor cells (control) and from the two strains containing the overexpression of the FABP4 gene.

### DESCRIPTION OF ILLUSTRATIVE EMBODIMENTS

The present disclosure overcomes challenges associated with ambient ionization MS analysis and imaging by providing an ambient ionization technique for spatially controlled, multiplex, comprehensive and sensitive molecular mass spectrometry (MS) imaging of surfaces, such as biological tissue samples. In some aspects, the present application provides an apparatus and technique for Micro Array Droplet Ionization (MADI). In certain aspects of MADI, miniaturized solvent droplets are deposited onto the sample surface, allowing molecular species to be extracted, followed by direct micro-aspiration and ionization of individual solvent-analyte droplets by a micro-capillary system. The molecular ions are characterized by high-performance MS analysis and 2D ion images are assembled. These techniques allow for high resolution imaging of samples that can be used to assess the changes in analytes across surface of a sample of interest.

By direct sampling of the solvent-analyte droplet, ion transmission is improved, thus improving overall sensitivity. Furthermore, not relying on analyte desorption and allowing for a longer solvent interaction time with the tissue surface, a larger mass range is expected to be achieved, expanding the possibilities for the analysis of larger biomolecules. To accurately dispense controlled droplet volumes, miniaturized liquid transfer tools such as a piezoelectric dispenser can be used.₁₂ Using a piezoelectric dispenser, the spatial resolution is accurately managed, as it depends strictly on dispensed volume and solvent-surface interaction, and higher spatial resolution is achieved by nano to pico droplet volumes. Finally, optimizing solvent polarity allows preferential extraction of desired target molecules, such as metabolites, lipids and proteins, with the potential for molecular multiplexing within tissue samples. Accordingly, the platform may be utilized to address several imaging applications including two relevant medical applications that elucidate the capabilities of MADI: (1) imaging of the tumor microenvironment in serous ovarian cancer tissue samples and (2) analysis of cerebrospinal fluid biopsies for brain tumors.

### I. An Apparatus of the Embodiments

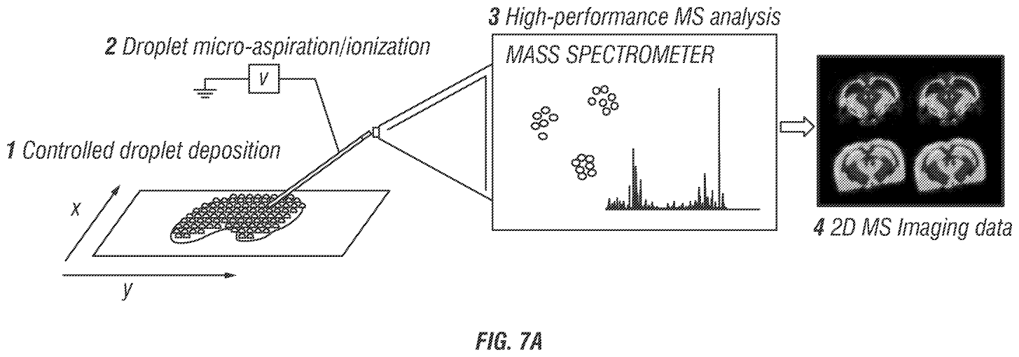

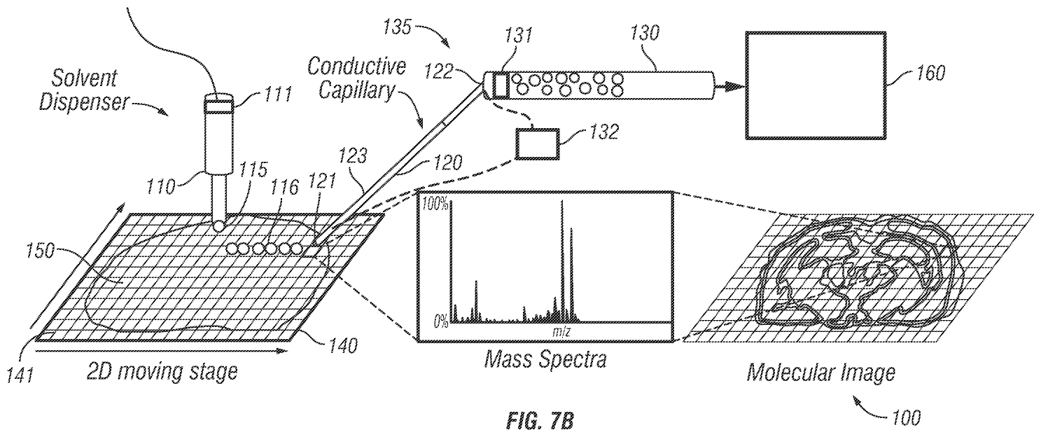

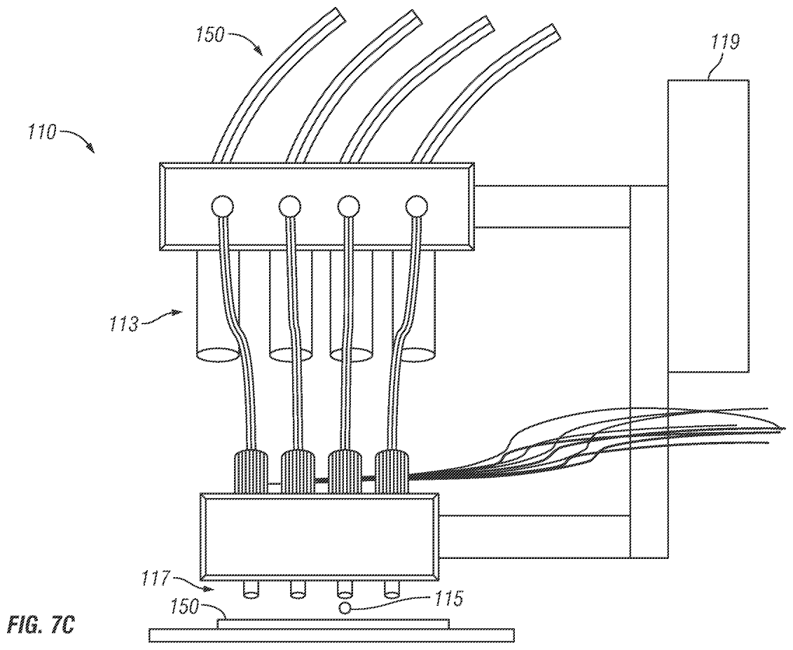

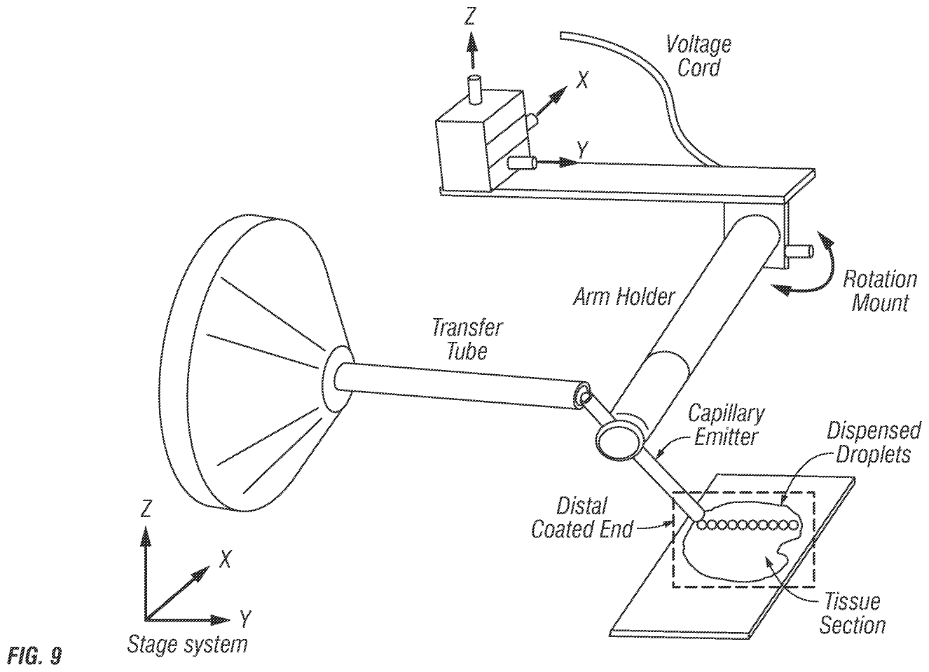

Referring now to FIG. 7B, an exemplary embodiment is illustrated of an apparatus 100 for producing samples for mass spectrometry analysis. In the embodiment shown, apparatus 100 comprises a solvent dispenser 110 and a collection system 135 comprising an electrically conductive conduit 120 (which can provide for sample ionization) and, optionally, a heated conduit (which can further ionize a sample) 130. In certain embodiments heated conduit 130 may be a heated mass spectrometer transfer tube. In certain embodiments, apparatus 100 comprises a sample retainer 140 configured to retain a sample 150 comprising one or more analytes. In exemplary embodiments, apparatus 100 may also comprise an actuator (not shown) configured to move sample retainer 140 in two (e.g. X-Y) or three (X-Y-Z) orthogonal directions such that droplets 115 are dispensed in a grid pattern 141 onto sample 150. In particular embodiments, droplets 115 can be spaced apart between 0.01 mm and 2.0 mm in grid pattern 141.

During operation of apparatus 100, solvent dispenser 110 is configured to dispense droplets 115 of solvent on sample 150 and electrically conductive conduit 120 is configured to transfer droplets 116 (comprising one or more analytes from sample 150 obtained via solvent droplets 115) to a mass spectrometer 160. It is understood apparatus 100 can be used in conjunction with any suitable mass spectrometer. During operation, an electrical potential can be applied to the electrically conductive conduit 120 to ionize the droplets 116 prior to transfer of droplets 116 to mass spectrometer 160.

In particular embodiments, solvent dispenser 110 may comprise a piezoelectric actuator 111 configured to dispense a precise volume of solvent in droplets 115. In specific embodiments, piezoelectric actuator 111 is configured to dispense droplets 115 that each comprise a volume between 5 and 50 picoliters (or more precisely between 10 and 30 picoliters). In one particular embodiment, each droplet 115 may have a volume of approximately 22 picoliters. During operation, sample retainer 140 can be moved in in between the dispensing of droplets 115 such that droplets 115 are dispensed in grid pattern 141 on sample 150.

After droplets 115 are dispensed on sample 150, electrically conductive conduit 120 can transfer droplets 116 (comprising one or more analytes) from sample 150 to mass spectrometer 160. As explained in further detail below, during operation of certain embodiments, analytes from sample 150 may be extracted by liquid solvation, followed by direct sampling of the analyte droplet 116 by the mass spectrometer 160.

In particular embodiments, electrically conductive conduit 120 may be coupled to a vacuum source in fluid communication with an inlet to mass spectrometer 160. In the embodiment shown, electrically conductive conduit 120 is also in fluid communication with heated conduit 130, which as previously mentioned, can together ionize droplets 116 prior to their analysis by mass spectrometer 160.

In the illustrated embodiment, electrically conductive conduit 120 comprises a first end 121 proximal to solvent dispenser 110 and a second end 122 distal from solvent dispenser 110. During operation, droplets 115 will enter electrically conductive conduit 120 via end 121 and exit electrically conductive conduit 120 via end 122. In particular embodiments, electrically conductive conduit 120 may be configured as a capillary tube comprising an electrically conductive material 123. In specific embodiments, electrically conductive material 123 may be a metal (e.g. platinum) coating proximal to first end 121. In certain embodiments, electrically conductive conduit 120 may be a silica tube comprising an outer diameter of approximately 360 micrometers (μm) and inner diameter of approximately 100 micrometers (μm).

In the illustrated embodiment, ionization of the sample is provided by a voltage source 132 is applied to the electrically conductive conduit 120. In further aspects, the system comprises a heating element 131. For example, the heating element 131 can be heated to a temperature between 250 and 350 Celsius (or more particularly, approximately 300 Celsius) in order to help with the desolvation of analyte droplets from 116. During operation of apparatus 100, voltage source 132 is applied to the electrically conductive conduit 120. Thus, a voltage differential between the electrically conductive conduit 120 and the mass spectrometer 160 (or the heated conduit 130) is provided. The ions in solution in droplets 116 are affected by the electric field from voltage source 132, causing charge separation and ion formation in a similar process to electrospray ionization (ESI). Free ions are formed in a heated transfer at elevated temperature and low pressure during the transfer to mass spectrometer 160.

As shown in FIG. 7c, in certain embodiments solvent dispenser 110 may comprise a piezoelectric picoliter dispenser with a plurality of pneumatic lines 112 configured to transport solvent out of reservoirs 113 and into dispensing tips 117. In particular embodiments, dispensing tips 117 may be glass piezoelectric tips. Controlling the voltage parameters provided to each glass piezoelectric tip, single droplets 115 of solvent in the picoliter range volume can be dispensed. The specific volume dispensed can depend on several factors, including for example, the solvent system used. To increase the resulting total volume, multiple droplets can be dispensed on the same spot (e.g. 30 drops, providing a cumulative volume of 0.65 nL). The diameter of the resulting droplets in contact with tissue sample 150 can thus be effectively controlled by changing the number of droplets dispensed per spot. Moreover, the distance between the droplets dispensed and between the tip and tissue sample is controlled by an actuator 119 (e.g. a 3-dimensional positioner), allowing to create droplet arrays with precise spatial control.

### II. Assay Methodologies

In some aspects, the present disclosure provides methods of determining imaging samples and detecting a molecular analyte signatures from a biological specimen. Samples for analysis can be from animals, plants or any material (living or non-living) that has been in contact with biological molecules or organisms. In some aspects, the samples are tissue sections, such as from a diseased organ.

Profiles obtained by the methods of the embodiments can correspond to, for example, proteins, metabolites, or lipids from analyzed biological specimens or tissue sites. Patterns may be determined by measuring the presence of specific ions using mass spectrometry and mapping them to their location in a sample.

As with many mass spectrometry methods, ionization efficiency can be optimized by modifying the conditions such as the solvent used, the pH, the gas flow rates, the applied voltage, applied temperature, and other aspects which affect ionization of the sample solution. In particular, the present methods contemplate the use of a solvent or solution which is compatible with biological tissue. Some non-limiting examples of solvent which may be used as the ionization solvent include water, ethanol, methanol, acetonitrile, dimethylformamide, an acid, or a mixture thereof. In some embodiments, the method contemplates a mixture of acetonitrile and dimethylformamide, or pure dimethylformamide solutions. The solvent mixtures may be varied to enhance the extraction of the analytes from the sample as well as increase the ionization and volatility of the sample. In some embodiments, the composition contains from about 5:1 (v/v) dimethylformamide:acetonitrile to about 1:5 (v/v) dimethyl-formamide:acetonitrile such as 1:1 (v/v) dimethylformamide:acetonitrile. In further aspects, the solvent can include components to enhance surface tension or surfactants.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Development of Droplet Array Aspiration Ionization

Enhanced lipid ionization by conductive silica capillary: In order to develop and design an optimal method for the ionization of lipid and metabolites directly from tissue samples, a total brain lipid solution of known concentration, containing primarily glycerophoshpholipids (GPs); was prepared in a 50:50 mixture of dimethylformamide (DMF) and acetonitrile (ACN). Polytetrafluoroethylene (PTFE) coated glass slides were used to deposit 0.5 μL droplets by manual pipetting. A blunt silica capillary (OD 360 μm-ID 100 μm) was carefully aligned to the inlet, and the front end MS negative pressure was used to aspirate the individual droplets. Successful ionization of lipid molecules was achieved by ion trap (IT) analysis in the negative ion mode. However, overall sensitivity and signal to noise ratio was low.

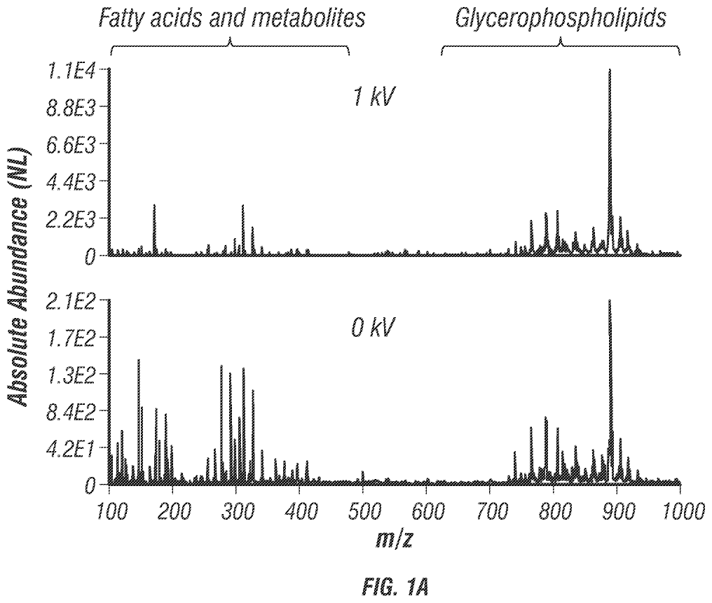

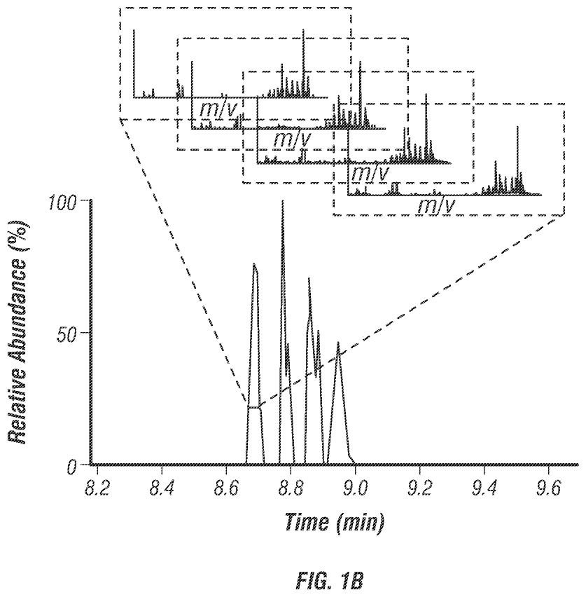

To improve ionization efficiency, an electric field was generated by applying a voltage bias to a platinum coated silica capillary. The capillary was coated at the distal end, away from the MS inlet. Consequently, the electrical contact was provided directly to the solvent-lipid droplet. FIG. 1A shows two representative mass spectra for ionization at 1 kV (top), versus 0 kV (bottom). The lipid species were characterized according to their mass to charge ratio (m/z). An increase in the spectrum absolute intensity (NL) was observed (2.1E2-1.1E4), with an average 40 fold increase for the 4 droplet replicates. As a result of the increased ionization efficiency, multiple MS spectra were obtained for an individual lipid droplet as shown in the total ion chromatogram (TIC) in FIG. 1B. The TIC represents the total abundance of all m/z species as a function of time, resulting from the aspiration of 4 analyte droplets at 1 kV. The averaging of the multiple spectra for each droplet provides a representative profile for each of the spots.

Further optimization experiments suggested that the voltage to trigger efficient ionization corresponded to values between 1-2 kV, with variability arising from multiple experimental parameters (i.e. distance and angle between capillary tip and MS inlet). Therefore, these results suggest careful voltage optimization is crucial for the development of an adequate mechanism for molecular analysis. Nevertheless, these results showed that the introduction of a potential bias between the capillary and the MS inlet significantly enhanced sensitivity by driving analyte ionization.

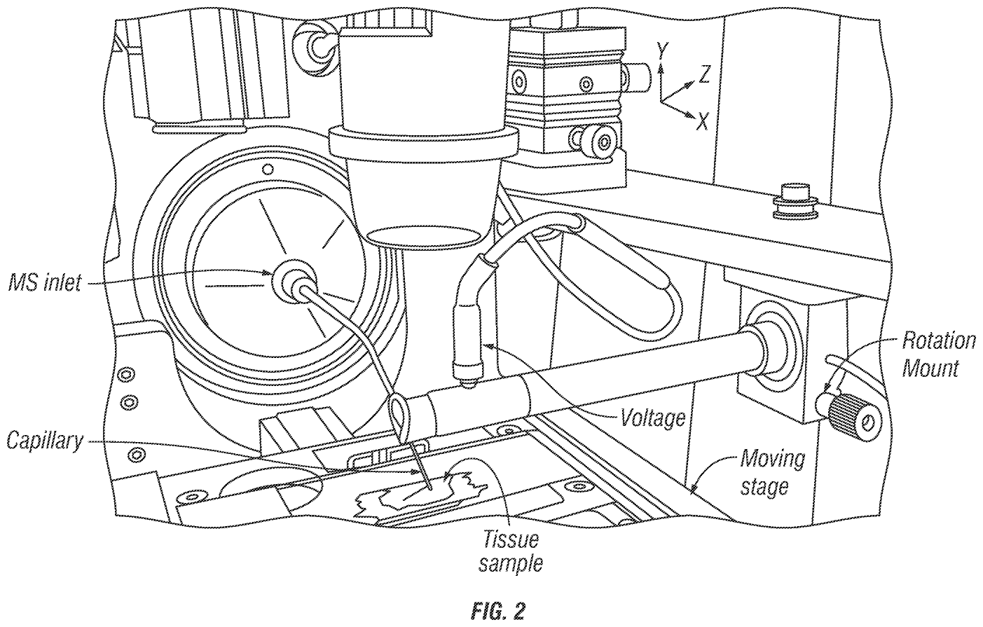

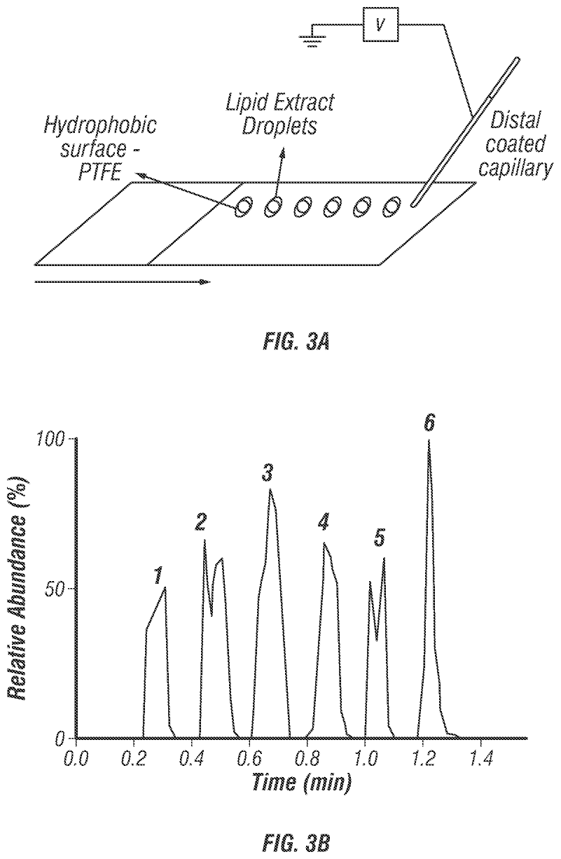

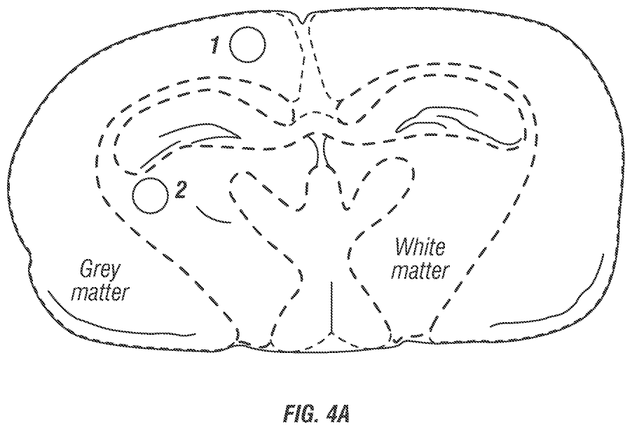

Development of a robust platform for reproducible analysis: Previous experiments indicated the importance of accurate and controllable alignment to achieve reproducible ionization of lipid droplets by DAAI. Thus, a robust arm holder using an XYZ stage for precise 3D positioning of the capillary tip was designed and assembled as shown in FIG. 2. Moreover, a rotation mount was utilized to control the angle of the capillary tip. The electrical contact was supplied to the conductive capillary by the source voltage from the mass spectrometer. Samples slides were placed on a 2D stage, moving at a constant rate, allowing for the continuous aspiration of individual droplets. To examine the reproducibility of the proposed system, standard lipid droplets (0.4 μL) were deposited on PTFE slides by manual pipetting and aligned in the z direction. The spatial coordinates were set for automatic aspiration, as shown in FIG. 3A. Lipid-solvent arrays of 6 droplets were analyzed by a high performance Orbitrap analyzer. FIG. 3B displays the TIC for six lipid droplets, where each rapid increase in relative abundance corresponds to an individual droplet. The average absolute intensity from the 6 droplet replicates was calculated to be of 1.71E5 NL with a relative standard deviation (RSD) of 12%. These values are within the range of variability reported for other ambient ionization techniques like DESI. However, fluctuations in relative abundances between lipid droplets were attributed to experimental pipetting error, changes in droplet shape, and misalignment of adjacent droplets, which could be addressed by the introduction of an automatic droplet dispenser. Nevertheless, these findings demonstrate the capabilities of DAAI to provide accurate and reliable molecular information representative of analyte composition and abundance in liquid solvent droplets.

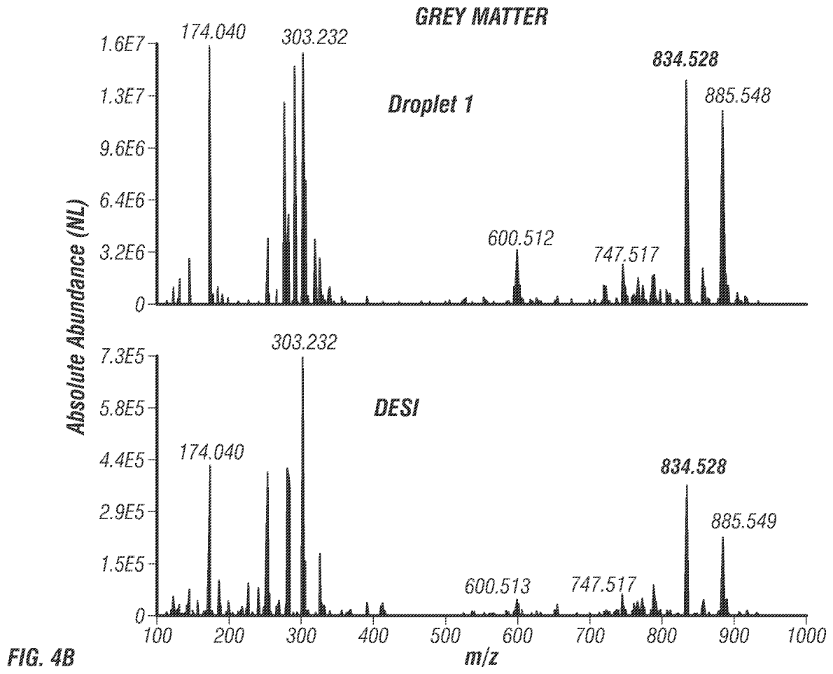

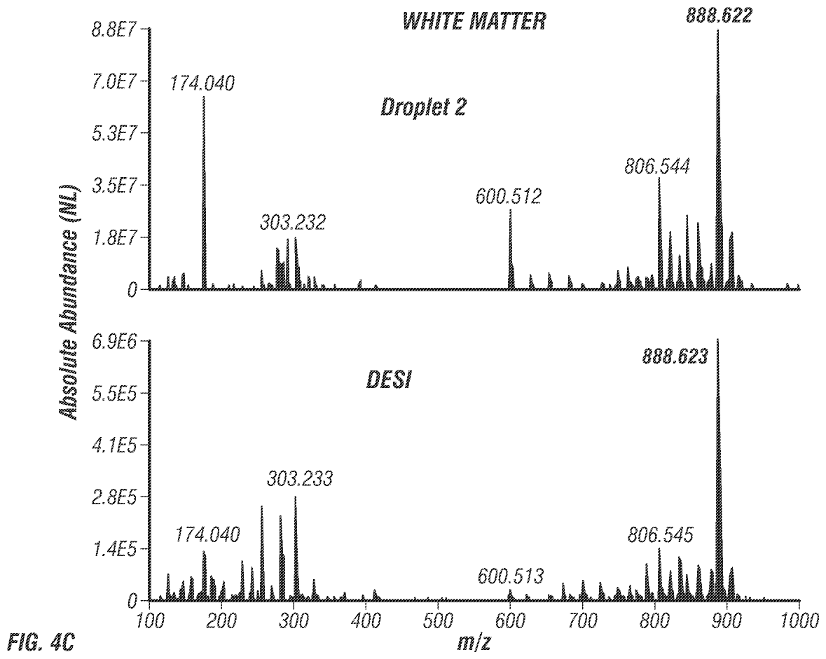

Lipid profiling from rat brain tissue sample: Rat brain has been widely used as a standard sample for molecular imaging as it presents two well defined molecular regions: white and grey matter. These two regions contain differences in cell composition and functionality and have been widely investigated in neurobiology (Olesen et al., 2003; Chittajallu et al., 2004). Previous analysis by DESI-MSI has shown distinctive lipid and metabolite species, allowing for clear identification of both areas (Wiseman et al., 2006). After successful and reproducible ionization of solvated lipid-droplets, DAAI was applied to the analysis of rat brain tissue. Smaller droplet volumes (0.2 μL) than those used in the previous sections were dispensed onto the tissue slide, to provide finer spatial resolution due to smaller droplet diameter. DMF was utilized as the dispensed solvent due to its high surface tension, successful lipid extraction, and low vapor pressure to avoid rapid evaporation at low volumes. Representative metabolic profiles from rat brain tissue were observed with outstanding sensitivity, which correlated to the droplet position onto the brain, and the results were compared to previous analyses performed by DESI (FIG. 4). Analysis of droplet 1, which was deposited on grey matter (FIG. 4A-B), presented characteristic molecular species for the region, where m/z 834.528, attributed to a glycerophosphoserine (PS) containing 40 carbons and 6 double bonds (40:6), was homogeneously distributed. In contrast, droplet 2 was deposited on the white matter region, providing characteristic molecular features defining of the area, such as the sulfatide lipid (ST) 24:1 attributed to m/z 888.622 (FIG. 4A-C). Other abundant molecular species were identified to be common glycerophospholipids, fatty acids and metabolites, providing similar metabolic profiles than those obtained by DESI analysis (FIGS. 4B and 4C). These results demonstrate the capabilities of DAAI for providing rich chemical information characteristic of molecularly distinct regions directly from biological tissue samples.

Controlled pico-droplet dispenser: Previous results demonstrated successful lipid ionization from solvated analyte droplets, both from lipid standard solution and from direct extraction from rat brain tissue. However, previous experiments utilized larger solvent droplets thereby limiting the spatial resolution achieved. To address these limitations in dispensing volume (0.1 μL), a piezoelectric picoliter dispenser was coupled to the DAAI setup for controlled deposition of nanodroplets onto rat brain tissue samples. The printhead assembly was mounted on a 3D positioner, to create arrays at controlled positions (Berglund et al., 2013).

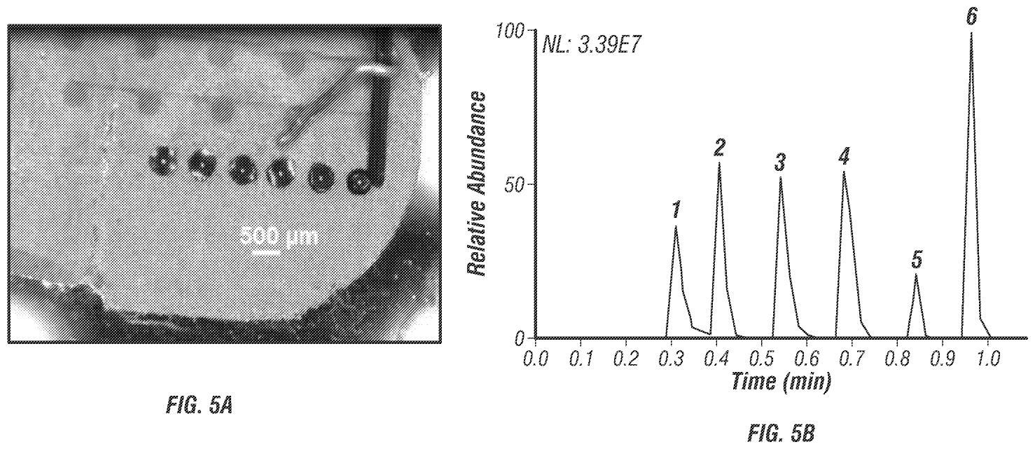

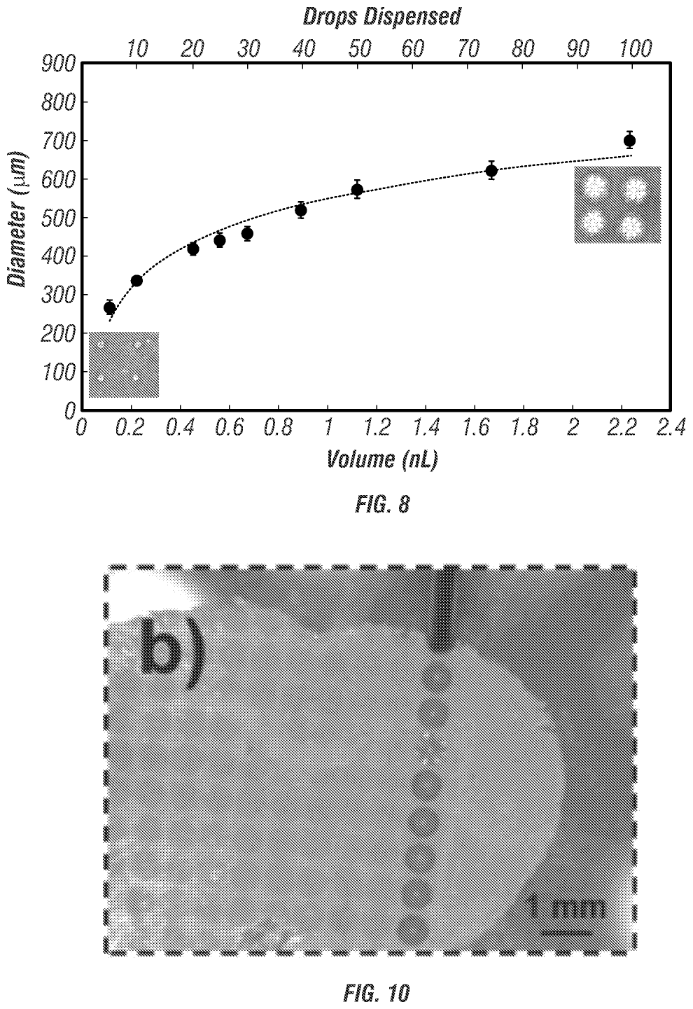

The smallest dispensed drops were calculated to be approximately 22 pL, based on a volume-mass calibration. Droplet size was controlled by the number of solvent drops dispensed per spot, such as 100 drops (˜2 nL) or 50 drops (˜1 nL), as shown in FIG. 8 for dimethylformamide droplets. FIG. 5 shows representative data from a 6 solvent droplet array of 1 nL deposited onto rat brain tissue. The droplet diameter, and hence spatial resolution, was measured to be ˜500 μm using a digital microscope. Successful ionization was attained, which provided rich chemical information characteristic for rat brain tissue.

By varying the number of drops dispensed, lipids and metabolites were extracted from the rat brain tissue sample at different spatial resolutions. Due to the high sensitivity performance of mass spectrometers, nanoliter solvent volumes were anticipated to be sufficient for analyte extraction and chemical analysis. Lipid and metabolite profiles were successfully obtained by MADI analysis of successive droplets of 20 drops each (˜0.4 nL), achieving 450 μm resolution.

### Example 2—Further Characterization of MADI

High spatial resolution ambient ionization mass spectrometry imaging by MADI: The capabilities of MADI for extraction and ionization of lipid and metabolites directly from tissue samples was described in Example 1. It was further sought to deposit successive droplet arrays from the dispenser to cover the entirety of the sample in order to map the distribution of lipid and metabolites across the tissue section. FIG. 7B displays the integration of the piezoelectric dispenser and aspiration/ionization setup onto a 2D moving stage for automatized imaging of biological tissue.

To construct the first molecular image by MADI, time and spatial parameters are evaluated. (1) Optimization of analyte extraction time prior to droplet evaporation allows for maximum extraction efficiency. As a result, solvent droplets are sequentially dispensed and extracted/ionized with precise time control as shown in FIG. 7B. (2) Aspiration time and data collection is timed by controlling the scan rates (μm/sec) for the moving stage, and the mass analyzer sampling time, to accurately correlate molecular information with spatial coordinates by data processing and imaging software. One of the main advantages of MADI over previous ambient ionization techniques is that it provides easily tunable spatial resolution of the chemical image. The piezoelectric dispenser capabilities mounted on a moving stage allow the sequential droplets to be deposited with minimal spatial distances, without causing droplet overlap. Thus, high spatial coverage is obtained. Moreover, by decreasing the size and volume of the dispensed solvent droplets, spatial resolution is accurately controlled. Surfactants or other additives, such as supercharging reagents, are added to increase surface tension of the solvent droplet for improved spatial resolution. Approaches for increasing substrate hydrophobicity, as well as different capillary tip geometries and solvent interaction will be explored to improve performance. By improving the droplet properties and ionization performance for smaller volumes (<0.4 nL) very fine control will be achieved to obtain high spatial resolutions (low μm scale).

Other considerations taken into account to improve performance and reproducibility are more accurately controlling the 2-D moving stage Y-axis position to accurately measure the distance between analyte-solvent droplet and the capillary tip. Further analysis of carry-over between adjacent solvent droplets is also performed to evaluate the need for incorporating washing steps to ensure optimal performance.

Comprehensive molecular analysis of biological samples: In order to understand the complexity of molecular processes entailing disease states in human disorders, a more comprehensive view into the biochemistry involved is necessary. Common demands to improve management of certain diseases, such as ovarian cancer, include the identification of biomarkers that can be targeted as therapeutics, combining the analysis of genes, proteins and lipids. DESI-MS has been employed to provide optimal conditions for analysis of small metabolites and lipids in both positive and negative ion mode polarities (Manicke et al., 2008). However, proteins have been widely underexplored with ambient ionization, due to inherent challenges of sample complexity, with lipids being preferentially desorbed and thus suppressing the detection of bigger biomolecules (Hsu et al., 2015). Although recent progress has been made, protein imaging under ambient conditions is still an ongoing challenge (Feider et al., 2016; Griffiths et al., 2016). Due to the proposed mechanism for extraction, not relying on desorption mechanisms, and allowing for longer solvent interaction time with the tissue surface, there is the potential for protein analysis at atmospheric pressure by MADI.

Analysis of positively charged lipids and proteins will be explored, which will entail optimization of adequate solvent systems for extraction of target analytes. Acetonitrile, methanol, chloroform and formic acid have been commonly used for analysis of proteins and lipids in the positive ion mode, due to good spray capabilities and extraction (Hsu et al., 2015). In order to achieve fine spatial resolution, well defined droplets are required. Consequently, solvents systems will be tested and modified to allow for optimal interactions with the biological surface. Since the volume dispensed depends on solvent properties, the parameters on the piezoelectric dispenser device will be adjusted to provide optimal spatial resolution.

After optimization of solvents for targeted analytes, comprehensive analysis of rat brain tissue samples will be performed, for lipid and proteins. Moreover, the multiplexing capabilities of MADI will be explored by changing solvent composition within the tissue section, targeting different analytes across different regions. Furthermore, the solvent properties and interaction with the tissue surface will be considered to provide the same spatial resolution across the sample. The implementation of MADI will allow for comprehensive analysis of tissue samples, providing valuable insights into molecular processes involved in biological disorders.

### Example 3—Clinical Applications

Tumor microenvironment in ovarian high-grade serous cancer: Ambient ionization MSI has been extensively used for the analysis of cancerous tissues, providing rich molecular descriptions informative of disease states (Eberlin et al., 2012; Eberlin et al., 2010). Currently, tissue biopsies are analyzed by skilled clinicians using light microscopy, which requires the staining of tissue slides by hematoxylin and eosin (H&E). This procedure allows differentiation of tissue types and morphological structures typical of cancer. The nuclei of the cell is stained in purple, while the cytosol, rich in connective tissue and free proteins is stained in pink. By using non-destructive solvent systems like in DESI-MS, this method may be compatible with this procedure, as the same tissue slide will be subjected to staining procedures and evaluated under the microscope (Eberlin et al., 2011). As a result, the spatial distribution of molecular species will be directly compared to cellular features present on the sample, which is essential to understand the changes observed in the molecular abundances.

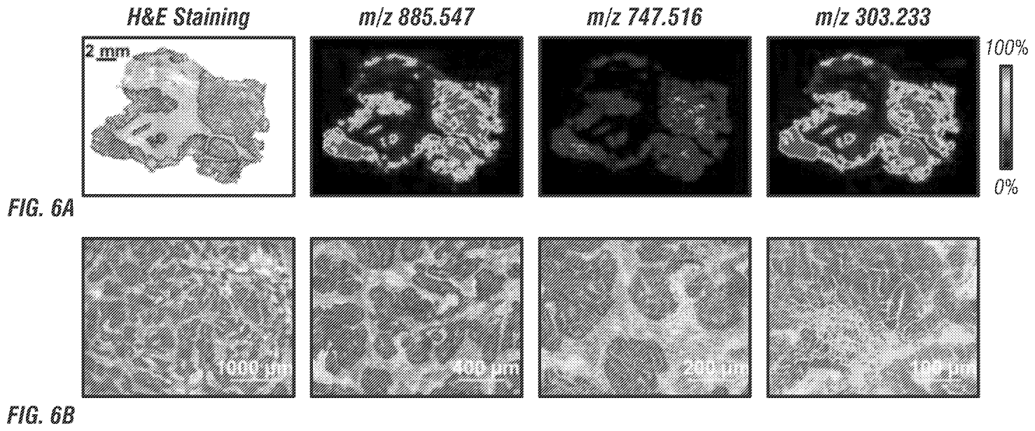

In a previous study, the metabolic and lipid profiles of ovarian serous cancers (SCs) has been investigated by DESI-MSI (Sans et al., 2016). FIG. 6A displays DESI-MS images for an ovarian high-grade SC tissue sample, and the corresponding staining showing the tumor regions outlined and stained in purple, due to the high concentration of nuclei. Areas of red intensity within the ion images represent highest (100%) and black lowest (0%) relative abundances. High relative abundances of certain species in the tumor regions, such as m/z 885.547, were observed compared to the surrounding connective tissue, which allowed visualization of the tumor clusters in high-grade SC.

High-grade SC is the most aggressive form of epithelial ovarian cancer and accounts for the majority of deaths in gynecological malignancies (Rosen et al., 2010). Recent studies have outlined the importance of identifying key players related to the development of the disease to improve patient outcome, by investigating early events and cellular features surrounding the tumor areas (Saad et al., 2010). FIG. 6B shows areas of tumor heterogeneity within the same tissue sample as in FIG. 6A at different magnifications to illustrate the differences in cell composition and architecture present in the tumor microenvironment. The previous analysis by DESI allowed clear visualization of the tumor clusters, but was limited to 200 μm in spatial resolution. Here, the high spatial resolution (in the low μm range) and multiplexing capabilities provided by MADI are exploited to explore the tissue heterogeneities observed in ovarian SC tissues. These analyses entail an improvement in analytical sensitivity, higher spatial resolution and control, and comprehensive molecular analysis (proteins and lipids) to help elucidate the micrometer heterogeneities. Moreover, a more in depth study into the tumor microenvironment, evaluating the role of stroma and connective tissue in contributing to tumorigenesis will be performed, which will allow for the investigation of factors responsible for early development of high-grade SC.

Analysis of cerebral spinal fluid for brain tumor diagnosis: For cancer diagnosis, tissue samples are usually collected during surgical procedures. However, the collection of samples from highly critical organs, such as in intracranial biopsies, entail higher risk. Consequently, blood or other bodily fluids in contact with the area of interest are usually analyzed. For brain tumors, cerebrospinal fluid (CSF) is commonly collected (Pan et al., 2015). As expected, these liquid samples present significantly lower cellular density than tissue samples, and consequently require a more sensitive method for analysis. Here, the enhanced sensitivity of MADI is utilized to analyze CSF samples. Since the spatial information from these samples is not essential, due to its previous liquid state where the analytes are distributed in solution, these analyses are not performed in the imaging mode but instead MADI is used as a profiling tool. The procedure requires minimal sample preparation, just spotting and drying the liquid samples on a glass slide. Then, the remaining cellular and molecular components are analyzed by MADI, depositing solvent droplets on the surface for aspiration/ionization. Adequate solvent systems are used to target multiple analytes within the sample. As a result, lipid and protein information characteristic from the sample are obtained, which are compared to healthy samples. The molecular species obtained characteristic of cancer in the CSF are used to develop statistical classifiers that can be used for diagnosis of the disease.

### Example 4—Methods of Droplet Formation and Analysis

Referring now to FIG. 9, utilizing a single piezoelectric tip, individual droplets were deposited onto the tissue surface, creating arrays of droplets in the y-direction.

An XYZ arm holder was coupled to a rotation mount to control the angle and positioning of the conductive emitter with respect to the MS inlet. The MADI setup was coupled to a piezoelectric picoliter dispenser for controlled deposition of dimethylformamide microdroplets. Individual droplets were aspirated using a blunt platinum coated silica capillary (OD 360 μm-ID 100 μm) aligned to the MS inlet. MADI imaging was performed by sequentially depositing and analyzing vertical lines of microdroplets from the tissue samples. A voltage bias was applied between the capillary and the MS inlet. A 2D moving stage (Prosolia Inc., IN) coupled to a QExactive mass spectrometer (Thermo Fisher Scientific, CA) was used, and images were built using RStudio.

To transport the droplets deposited onto the tissue sample to the MS, a silica capillary (OD 360 μm, ID 100 μm) was aligned to the MS inlet using an arm holder coupled to an XYZ stage and rotation mount to control the positioning and angle of the emitter with respect to the transfer tube, as shown in FIGS. 9 and 10. An extended transfer tube was used to enable a wider range of motion in the y-direction, also allowing to transfer analytes from the capillary emitter to the heated part of the inlet MS tube. Taking advantage of the differential between the ambient pressure (˜1.0 bar) and the fore vacuum pressure provided by the front end of the instrument (1.6-1.9 mbar, Q Exactive Orbitrap), droplets were sequentially aspirated and transported to the MS after being placed in contact with the distal end of the emitter. The silica emitter was platinum coated at the distal end, allowing the application of a voltage bias between the MS inlet and the end of the capillary. The end of the capillary proximal to the transfer tube was left uncoated to prevent electrical arcing or discharge due to release of electrons from both electrodes at short distances.

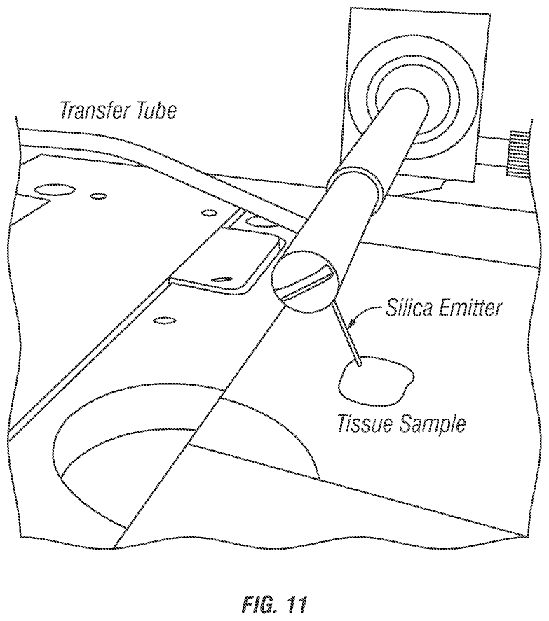

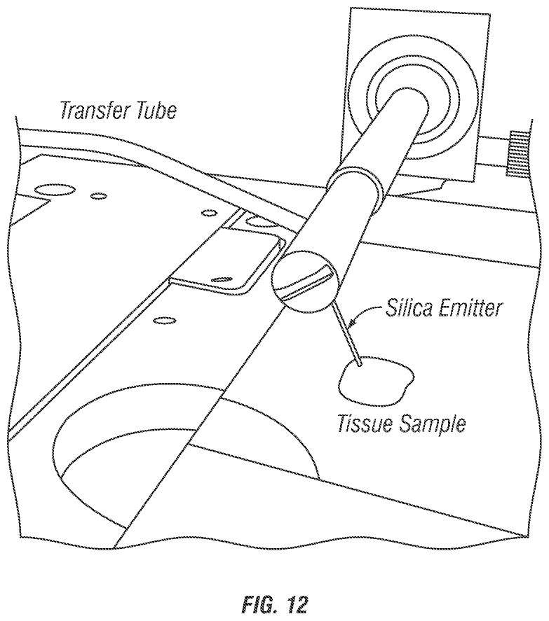

By inserting the capillary emitter into the transfer tube, the analyte is introduced directly into the MS system for analysis, avoiding any sample loss. The inventors hypothesize that droplet formation and desolvation occurs due to the pressure drop and temperature increase between atmospheric environment and the inlet tube, as reported in solvent assisted inlet ionization (SAID and other inlet ionization methods (Pagnotti, et al. 2011, McEwen et al. 2010, Trimpin et al. 2010). Moreover, the application of a voltage differential enhances charge separation inside the solvated droplets, inducing the formation of free ions and thus increasing ionization efficiency, a mechanism characteristic of electrospray ionization (ESI) and also described in electrosprayed inlet ionization (ESII), where ionization by SAII is improved by voltage application (Pagnotti et al. 2012, Konermann et al. 2013). However, discrete and small volumes of analyte are introduced for analysis by MADI after extraction of molecular species from tissue surfaces, while ESI and ESII utilize a continuous flow of solvent directly from liquid samples. Similar lipid profiles, in terms of species detected and relative abundance, were observed by analyzing a mouse brain lipid extract by MADI and ESI as shown in FIGS. 11 and 12. For this experiment, extract droplets were deposited on a glass slide and analyzed sequentially by MADI, while ESI analysis was performed by direct infusion of the brain extract solution. This result suggests that ion formation by MADI is induced through a similar mechanism to that of ESI or ESII; by droplet formation, desolvation and charge separation.

### MADI-MS Optimization for Lipid Analysis in the Negative Ion Mode

The analysis of metabolites, fatty acids and complex lipids from tissue sections by MS imaging has gained increasing attention in the last two decades, as new insights and developments have consistently indicated the important role of lipid metabolism in a variety of human diseases (Wenk, 2005). MS analysis of deprotonated lipid molecules in the negative ion mode provides a wide variety of fatty acid and glycerophospholipid (GP) species, negatively charged at pH 7 due to the carboxylic acid and phosphate groups, respectively. Sphingolipids (SP), such as ceramides, can also be detected in the negative ion mode by chlorine adduction. On the other hand, positive ion mode analysis of lipid species provides mass spectra mostly characterized by glycerophosphocholine (PC) species. Thus, to achieve greater molecular diversity, the capabilities of MADI-MS for lipid analysis directly from tissue sections were first explored in the negative ion mode.

Commonly used solvents for lipid extraction and analysis in the negative ion mode include, dimethylformamide (DMF), water, ethanol, methanol, acetonitrile, and chloroform (Eberlin et al. 2011). Based on the unique characteristics of MADI-MS, specific properties were required for the solvent system of choice. Due to its high lipid solubility, high surface tension, limited adhesion to the tissue samples, and low vapor pressure, dimethylformamide (DMF) was selected as the optimal solvent system for MADI-MS lipid analysis in the negative ion mode. The high surface tension of DMF (37 mN/m) allowed the formation of droplets at high contact angles. Even though water is characterized by a higher surface tension (73 mN/m), the mild hydrophilicity of the tissue samples entailed droplet spreading and lower contact angles. Finally, the low vapor pressure prevented rapid evaporation of solvent-analyte droplets prior to analysis. By controlling the number of DMF drops dispensed, droplet diameters ranging from 300 to 500 μm were achieved upon dispensing onto tissue samples. A logarithmic relationship between volume dispensed and droplet diameter was observed. With this platform, controlled solvent volumes were dispensed onto tissue samples, providing exceptional control over the area sampled, and thus, the spatial resolution.

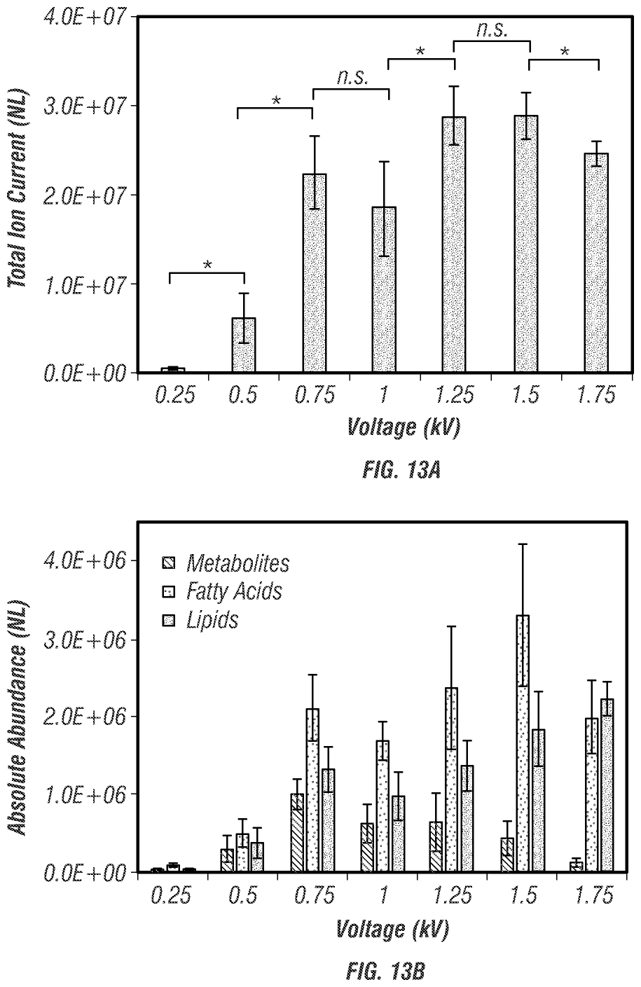

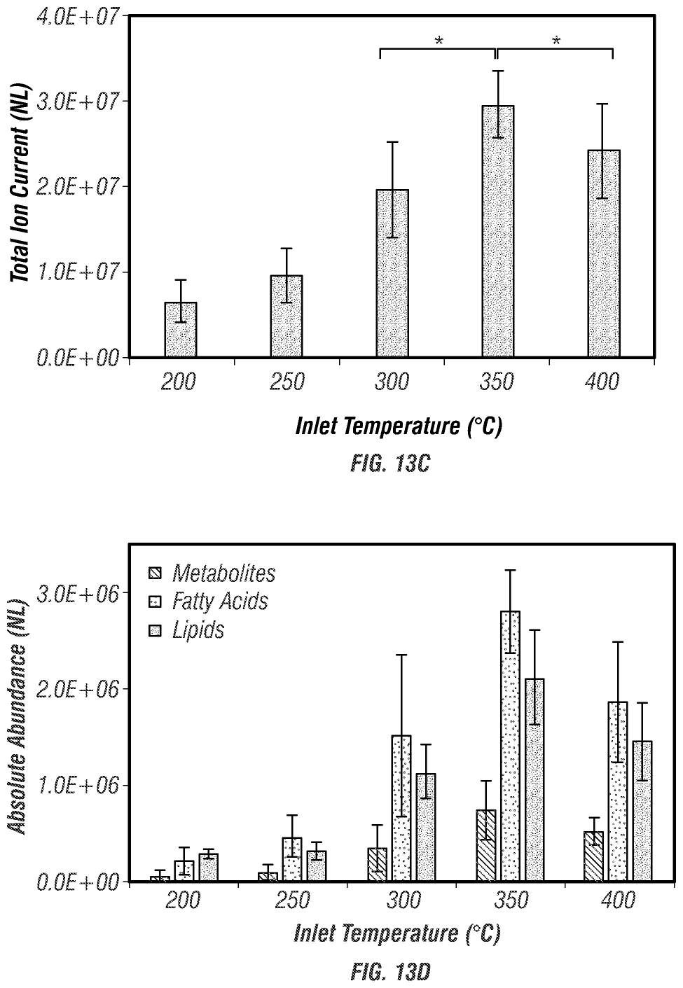

To provide optimal detection of lipid species, various parameters within the MADI-MS system were also evaluated, such as emitter positioning, inlet temperature, and source voltage. Best performance was observed by placing the capillary emitter just inside the extended transfer tube, aligned to the center of the tube orifice in both the Z and X directions, and at an angle of approximately 60-70° relative to the moving stage, allowing improved transfer of the analyzed droplets to the MS system. To quantify the effect of inlet temperature and source voltage on the abundance of the detected species, replicate arrays of 40-drop droplets (approximately 0.9 nL) were dispensed onto tissue sections and analyzed at different parameters. Tissue sections from a brain homogenate were used to minimize tissue heterogeneities. Optimal performance was achieved at an inlet temperature of 350° C., with higher TIC values observed by increasing inlet temperature, as shown in FIG. 13 panel (a). These results suggest that higher temperatures enhance ionization efficiency by potentially facilitating solvent evaporation. A significant decrease on the TIC was observed at 400° C., possibly due to the degradation of metabolic species at such high temperatures. A similar trend was also observed by plotting the combined absolute abundance detected for different types of molecules, such as metabolites, fatty acids and lipids as shown in FIG. 13 panel (b).

Although the application of an external electric field was not necessary to produce ions above S/N for larger (>50 nL) droplets by MADI-MS, the application of a potential bias improved performance of the method for smaller droplets (<1 nL) needed for imaging applications at the desirable spatial resolution. At these smaller volumes, applying a voltage (0.25 kV) to the capillary emitter was required to produce detectable signal, indicating that inlet ionization alone was not sufficient to ionize small and discrete volumes of solvated analyte. Improvements in TIC values were observed by increasing the applied voltage to 1.25-1.5 kV as shown in FIG. 13 panel (c), while smaller TIC values were detected at 1.75 kV. Assessment of the changes in absolute abundance measured for certain metabolites, fatty acids and lipids species, as shown in FIG. 13 panel (d), revealed that the decrease in TIC at 1.75 kV could be attributed to the detection of lower ion currents at m/z values corresponding to metabolite and fatty acid species, suggesting that smaller molecules might lose stability at higher electric fields.

### MADI-MS Imaging of Mouse Brain Tissue Samples

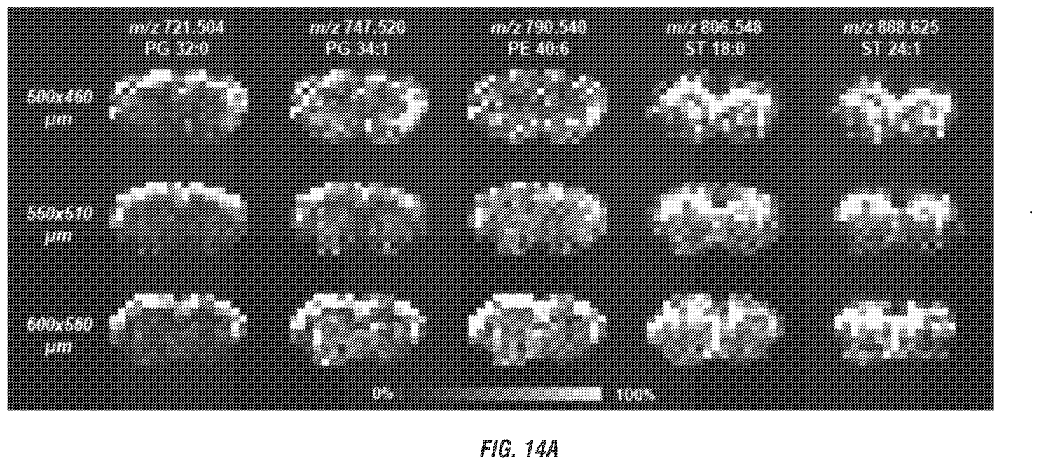

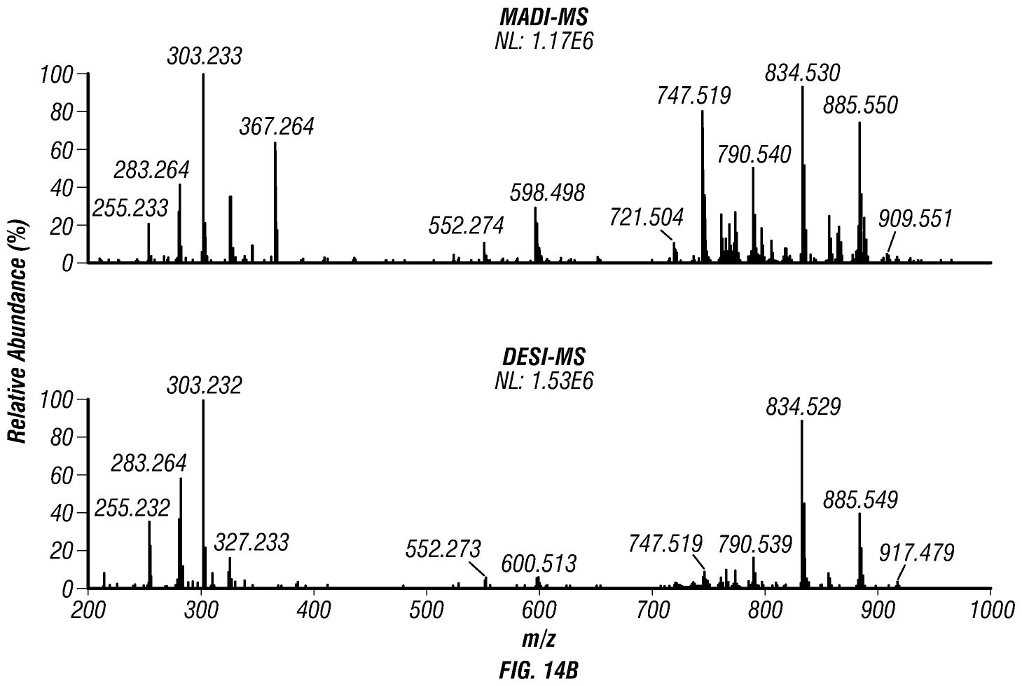

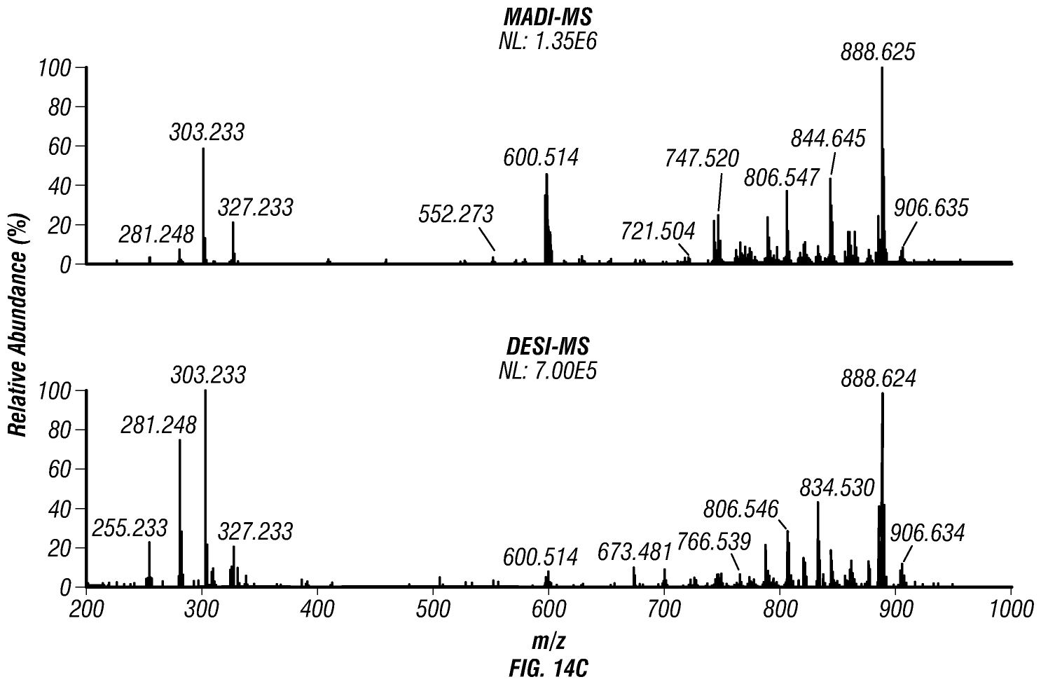

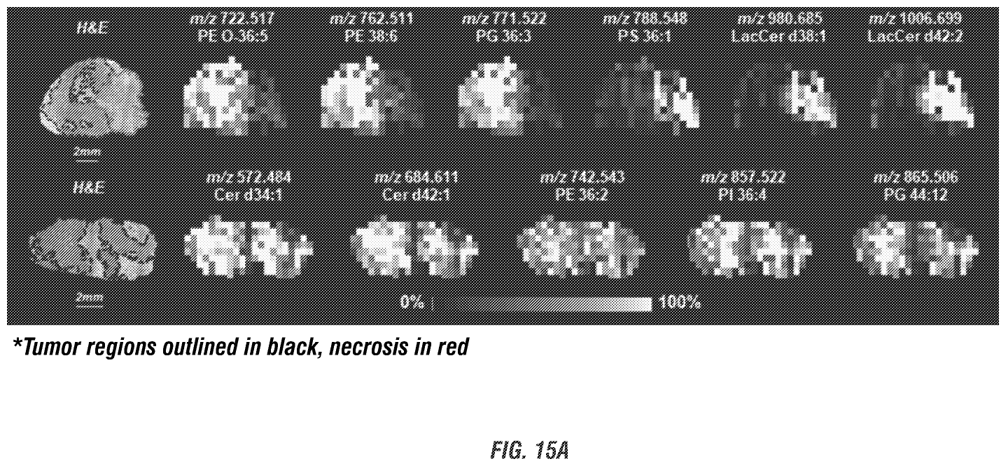

Serial sections of mouse brain tissue samples were analyzed by MADI-MS images at various spatial resolutions by changing dispensed solvent volume. Row and column spacing was added (140 μm, ˜100 μm, respectively), to avoid droplet overlap, resulting in spatial resolutions of 500×460 μm, 550×510 μm, and 600×560 μm. Characteristic profiles and ion images were obtained, allowing the depiction of grey and fine white matter regions as shown in FIG. 14 panel (a). Similar distributions of the detected lipid species were observed at the three different spatial resolutions, demonstrating method robustness and reproducibility. As expected, improved definition of white matter features was achieved at higher spatial resolutions, where sulfatide species, such as ST 18:0 (m/z 806.548) and ST 24:1 (m/z 888.625), are commonly located within brain tissues. These results show the first demonstration of MADI MS for tissue imaging as well as the capabilities of MADI-MS to effectively control the imaging spatial resolution.

Apart from the visualization of tissue heterogeneity by MADI-MS imaging, rich metabolite, fatty acid and lipid information was also observed from MADI-MS spectra, offering different molecular profiles than those typically observed by DESI-MS analysis. Interestingly, many complex lipid species, including m/z 598.498 (ceramide—Cer d36:2), m/z 721.504 (glycerophosphoglycerol—PG 32:0), m/z 747.52 (PG 34:1), and m/z 790.54 (glycerophosphoethanolamine—PE 40:6), displayed higher relative abundances within MADI spectra from grey matter brain regions (—30-80%) compared to DESI spectra (<20-10%), as shown in FIG. 14 panel (b). Differences in the lipid species detected and their relative abundances were also detected between MADI and DESI spectra from white matter regions. Moreover, higher total ion currents were generally detected by MADI-MS from white matter regions, when compared with an optimal DESI system at the same spatial resolution as shown in FIG. 14 panel (c). These results suggest that MADI-MS can offer comprehensive and sensitive molecular analysis of tissue samples by coupling an effective liquid extraction with direct ionization through a conductive silica capillary.

### Sensitive Analysis and Imaging of Cancerous Tissue Samples by MADI-MS

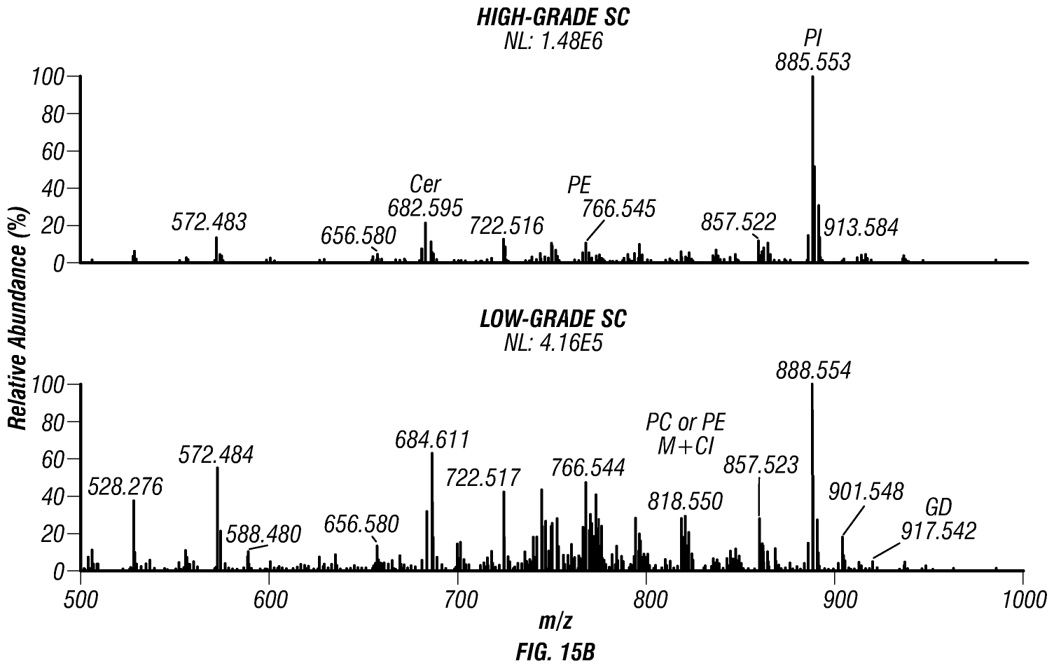

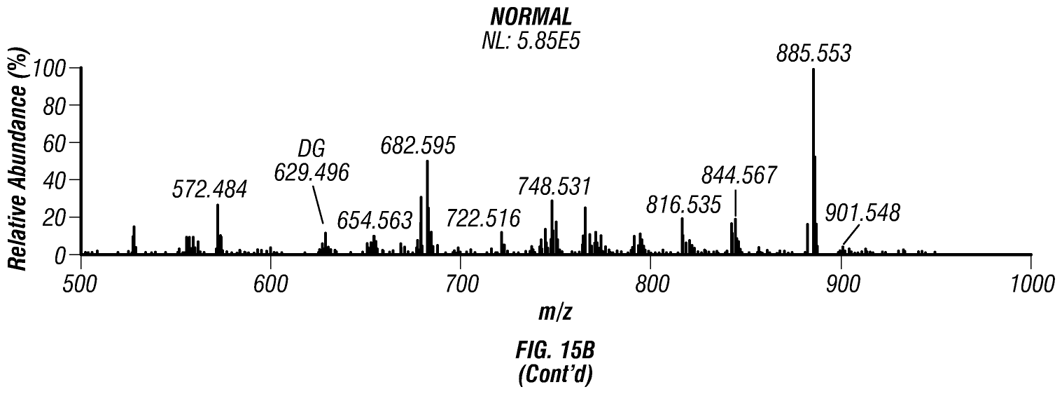

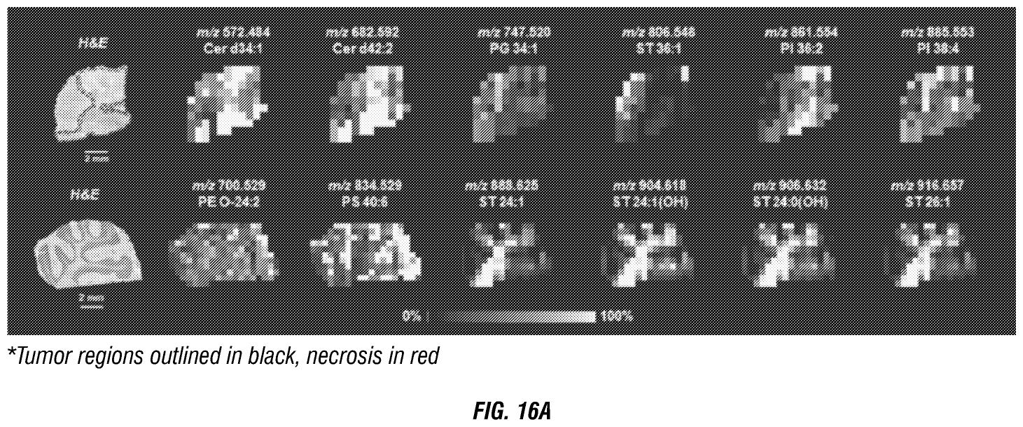

MADI-MS was also applied to analyze normal and tumor human ovarian tissue samples (n=5). Heterogeneous distributions for a variety of different ions where observed by MADI-MS, correlated to histological differences within the tumor tissue samples, as shown in FIG. 15 panel (a). As observed in previous studies utilizing ambient ionization to characterize ovarian tumor samples, an overall higher abundance of lipid species was detected from areas corresponding to high tumor cell concentrations (delineated in black), compared to the surrounding connective tissue (Sans et al. 2017). Characteristic ion images and profiles were also observed from necrotic regions (outlined in red), presenting high relative abundances of lactosylceramide (LacCer) species, such as LacCer d38:1 (m/z 980.685) or LacCer d42:2 (m/z 1006.699). FIG. 15 panel (b) shows representative MADI-MS spectra from tumor samples, including high-grade serous carcinoma (SC) and low-grade SC ovarian samples, and normal ovarian tissue. A variety of lipid species were detected at high relative abundance within the mass spectra, including deprotonated and chlorine adducts from Cer, PE, PC, diacylglycerol (DG), and glycerophosphoinositol (PI) species, as well as doubly charged ganglioside (GD) lipids. Qualitative differences in species detected and their abundances were observed between tissue type, showcasing the capabilities of MADI-MS to obtain characteristic molecular information directly from tissue samples.

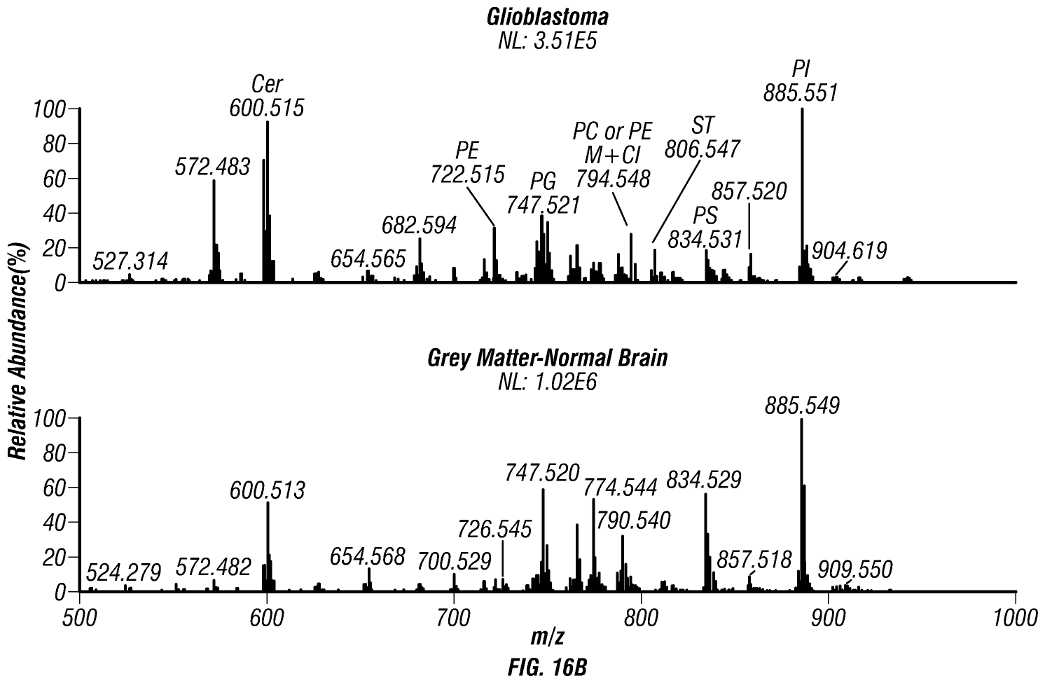

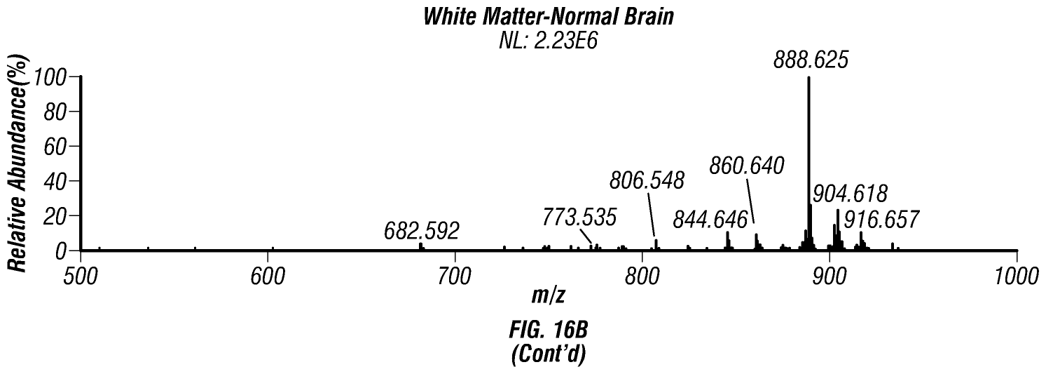

To demonstrate the wide applicability of MADI-MS for imaging and sensitive analysis of cancer and human tissue, a glioblastoma tumor sample and a normal human brain sample were analyzed. Heterogeneous features were observed within the ion images obtained from the glioblastoma tissue sample. Pathological evaluation revealed a mixture of tumor and necrotic regions, as shown in the outlined hematoxylin & eosin (H&E) stained tissue sample in FIG. 16 panel (a) (top). Differences in the lipid profiles and thus ion images were observed between areas containing tumor cells and the necrotic regions. For example, ceramide species were detected at higher relative abundances from necrotic regions, while other lipids, such as PG 34:1 (m/z 747.520) or ST 36:1 (m/z 806.548) were detected at higher relative abundances within the tumor region. Interestingly, a depletion of a variety of lipid species, such as PI 36:2 or PI 38:4 were observed in the tumor area. Previous studies utilizing DESI-MS to investigate brain tumors have also reported an overall decrease in lipid abundance in tumor tissue compared to normal tissue (Eberlin et al. 2010). MADI-MS ion images from normal brain tissue allowed excellent visualization of white matter regions, characterized by the distribution of sulfatide species (e.g. ST 24:1, ST 24:1(OH), ST 24:0(OH), and ST 26:1) forming branching architectures within the tissue sample as shown in FIG. 16 panel (a) bottom row. Other species, such as the molecular ion at m/z 700.529, tentatively identified as PE O-24:2, displayed a homogeneous distribution, while other species, such as PS 40:6 (m/z 834.529), offered a complementary distribution to the sulfatide species, with a higher relative abundance observed from the grey matter region. These differences in molecular composition between glioblastoma tissue, and grey and white matter from normal brain, were also visualized from the mass spectra acquired by MADI-MS analysis shown in FIG. 16 panel (b). A variety of lipid species were detected at high relative abundances, and were tentatively identified as Cer, PE, PG, PC, ST, PS, and PI, among other complex lipid species. These results demonstrate the capabilities of MADI-MS for spatially controlled imaging, and sensitive, comprehensive analysis from complex and heterogeneous tissue samples.

### Other Applications for Analysis of Biological Samples

Due to the capabilities of MADI-MS for rapid, sensitive and comprehensive analysis, this method can also be applied to investigate a variety of biological samples, not limited to tissues sections only. Thus, here the inventors demonstrate the use of MADI-MS to analyze cell samples deposited and dried onto a glass slide. Contrary to other traditional methods to analyze cell samples by MS, requiring the lengthy and labor-intensive extraction and purification of cell extracts to investigate their molecular composition, MADI-MS provides the ability to extract analytes directly and rapidly from the dried cell pellet, followed by efficient transport and ionization for MS analysis. As the distribution of the molecular species is not necessary in this application, solvent volumes can be dispensed onto the sample manually, using a pipette. The resulting volume containing extracted analytes can then be aspirated and analyzed by MADI-MS.

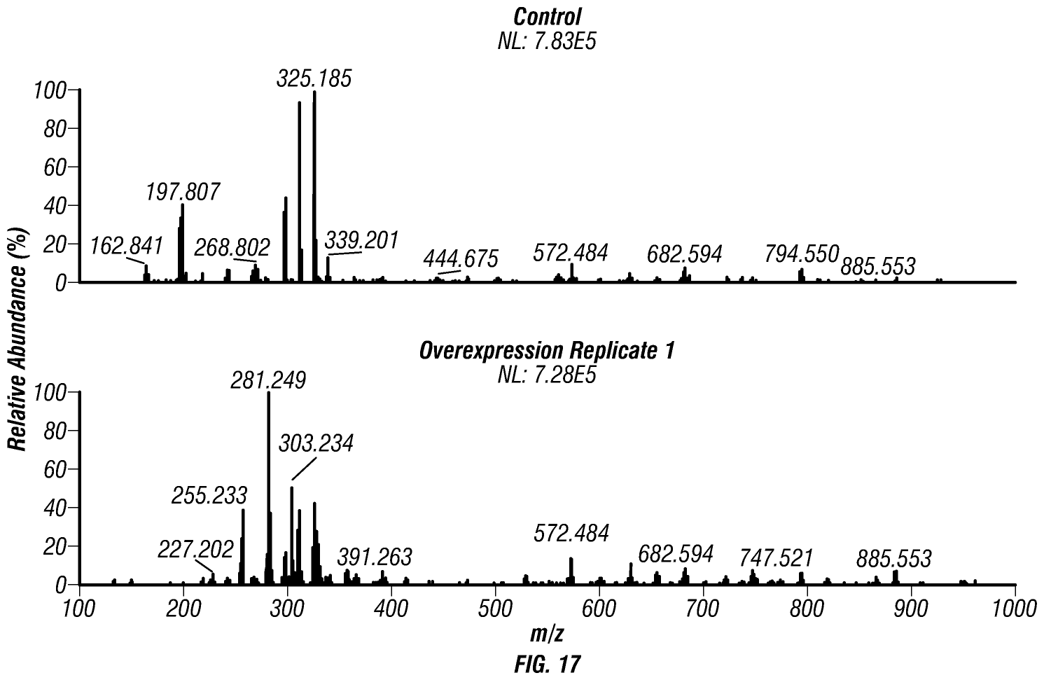

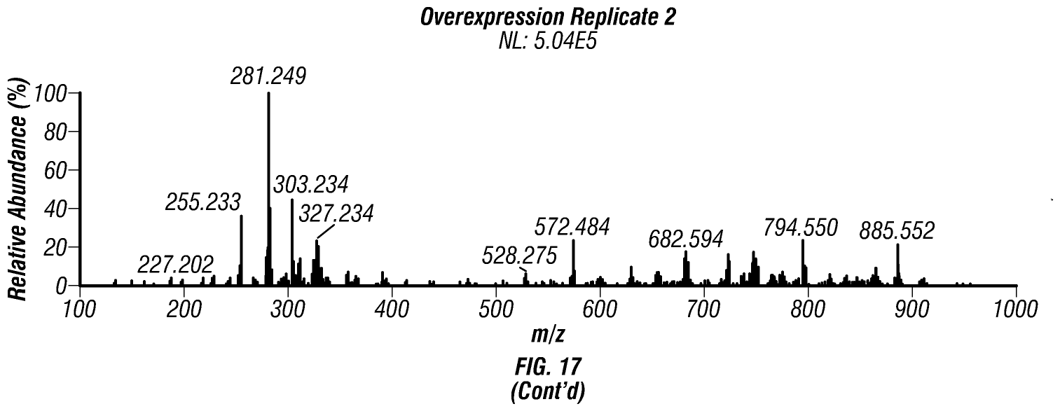

To demonstrate feasibility and successful analysis of cell samples, the inventors applied MADI-MS to evaluate various human ovarian cell pellets. Rich molecular information, including various metabolites, fatty acid and lipid species were detected from MADI-MS spectra. FIG. 17 provides representative profiles obtained from the analysis of tumor ovarian cells (control) and two replicates of a genetically modified strain of human ovarian tumor cells, with the overexpression of the fatty acid binding protein (FABP4) gene. Notable differences in fatty acid composition were observed between the different cell lines, with a higher relative abundances of fatty acid species, such as m/z 281.249 (fatty acid—FA 18:1), m/z 303.324 (FA 20:4), and m/z 327.234 (FA 22:6) detected from the samples with FABP4 overexpression. A variety of lipid species were also detected in the spectra from the m/z 500 to 1000 range. These results showcase the capabilities of MADI-MS to provide sensitive and reproducible analysis of cell samples, resulting in metabolic profiles representative of cell composition.

Current efforts are directed towards the application of MADI-MS for positive ion mode lipid analysis as well as automation of the imaging platform to allow for sequential deposition and aspiration of individual droplets.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Badu-Tawiah et al., Chemical Aspects of the Extractive Methods of Ambient Ionization Mass
Spectrometry. Annu Rev Phys Chem, 64, 481-505, 2013.
Berglund et al., Screening of transition and post-transition metals to incorporate into copper oxide and copper bismuth oxide for photoelectrochemical hydrogen evolution. Phys Chem Chem Phys, 15 (13), 4554-4565, 2013.
Chittajallu et al., NG2-positive cells in the mouse white and grey matter display distinct physiological properties. The Journal of Physiology, 561 (1), 109-122, 2004.
Cooks et al., Ambient mass spectrometry. Science, 311 (5767), 1566-1570, 2006.
Didangelos et al., The -omics era: Proteomics and lipidomics in vascular research. Atherosclerosis, 221 (1), 12-17, 2012.
Eberlin et al., Cholesterol Sulfate Imaging in Human Prostate Cancer Tissue by Desorption
Electrospray Ionization Mass Spectrometry. Anal. Chem., 82 (9), 3430-3434, 2010.
Eberlin et al., Classifying human brain tumors by lipid imaging with mass spectrometry. Cancer Res, 72 (3), 645-54, 2012.
Eberlin et al., Molecular assessment of surgical-resection margins of gastric cancer by mass-spectrometric imaging. Proc Natl Acad Sci USA, 111 (7), 2436-41, 2014.
Eberlin et al., Nondestructive, histologically compatible tissue imaging by desorption electrospray ionization mass spectrometry. Chembiochem, 12 (14), 2129-32, 2011.
Feider et al., Ambient Ionization and FAIMS Mass Spectrometry for Enhanced Imaging of Multiply
Charged Molecular Ions in Biological Tissues. Anal. Chem., 88 (23), 11533-11541, 2016.
Fischer et al., Hematoxylin and eosin staining of tissue and cell sections. CSH protocols 2008, pdb.prot4986, 2008.
Giesen et al., Highly multiplexed imaging of tumor tissues with subcellular resolution by mass cytometry. Nat Methods, 11 (4), 417-+, 2014.
Griffiths et al., LESA FAIMS Mass Spectrometry for the Spatial Profiling of Proteins from Tissue. Anal. Chem., 88 (13), 6758-6766, 2016.
Guenther et al., Spatially resolved metabolic phenotyping of breast cancer by desorption electrospray ionization mass spectrometry. Cancer Res 75 (9), 1828-37, 2015.
Harris et al., Ambient Sampling/Ionization Mass Spectrometry: Applications and Current Trends. Anal. Chem., 83 (12), 4508-4538, 2011.
Hsu et al., Imaging of Proteins in Tissue Samples Using Nanospray Desorption Electrospray Ionization Mass Spectrometry. Anal Chem, 87 (22), 11171-5, 2015.
Ifa and Eberlin, Ambient Ionization Mass Spectrometry for Cancer Diagnosis and Surgical Margin Evaluation. Clin Chem, 62 (1), 111-2., 2016.
Ifa et al., Quantitative analysis of small molecules by desorption electrospray ionization mass spectrometry from polytetrafluoroethylene surfaces. Rapid Commun Mass Sp, 22 (4), 503-510, 2008.
Manicke et al., Desorption electrospray ionization (DESI) mass Spectrometry and tandem mass spectrometry (MS/MS) of phospholipids and sphingolipids: Ionization, adduct formation, and fragmentation. J Am Soc Mass Spectr, 19 (4), 531-543, 2008.
McDonnell and Heeren, Imaging mass spectrometry. Mass Spectrom Rev 26 (4), 606-643, 2007.
Olesen et al., Combined analysis of DTI and fMRI data reveals a joint maturation of white and grey matter in a fronto-parietal network. Cognitive Brain Research, 18 (1), 48-57, 2003.
Pan et al., Brain Tumor Mutations Detected in Cerebral Spinal Fluid. Clinical Chemistry, 61 (3), 514-522, 2015.
Rosen et al., Morphological and molecular basis of ovarian serous carcinoma. J Biomed Res, 24 (4), 257-63, 2010.
Saad et al., Microenvironment and pathogenesis of epithelial ovarian cancer. Horm Cancer, 1 (6), 277-90, 2010.
Sans et al., Metabolic Markers and Statistical Prediction of Serous Ovarian Cancer Aggressiveness by Ambient Ionization Mass Spectrometry Imaging. Cancer Res 2016.
Seeley and Caprioli, MALDI imaging mass spectrometry of human tissue: method challenges and clinical perspectives. Trends Biotechnol, 29 (3), 136-43, 2011.
Vaughan et al., Rethinking ovarian cancer: recommendations for improving outcomes. Nat Rev Cancer, 11 (10), 719-725, 2011.
Venter et al., Droplet dynamics and ionization mechanisms in desorption electrospray ionization mass spectrometry. Anal. Chem., 78 (24), 8549-8555, 2006.
Wiseman et al., Tissue imaging at atmospheric pressure using desorption electrospray ionization (DESI) mass spectrometry. Angew Chem Int Edit 45 (43), 7188-7192, 2006.
Zhang et al., Cardiolipins are biomarkers of mitochondria-rich thyroid oncocytic tumors. Cancer Res 2016.
Pagnotti, V. S.; Chubatyi, N. D.; McEwen, C. N. Anal Chem 2011, 83, 3981-3985.
McEwen, C. N.; Pagnotti, V. S.; Inutan, E. D.; Trimpin, S. Anal. Chem. 2010, 82, 9164-9168.
Trimpin, S.; Inutan, E. D.; Herath, T. N.; McEwen, C. N. Mol Cell Proteomics 2010, 9, 362-367.
Pagnotti, V. S.; Chakrabarty, S.; Harron, A. F.; McEwen, C. N. Anal Chem 2012, 84, 6828-6832.
Konermann, L; Ahadi, E.; Rodriguez, A. D.; Vahidi, S. Anal. Chem. 2013, 85, 2-9.
Wenk, M. R. Nat Rev Drug Discov 2005, 4, 594-610.
Eberlin, L. S.; Ferreira, C. R.; Dill, A. L.; Ifa, D. R.; Cheng, L.; Cooks, R. G. Chembiochem 2011, 12, 2129-2132.
Sans, M.; Gharpure, K.; Tibshirani, R.; Zhang, J.; Liang, L.; Liu, J.; Young, J. H.; Dood, R. L.; Sood, A. K.; Eberlin, L. S. Cancer Res 2017, 77, 2903-2913.
Eberlin, L. S.; Dill, A. L.; Golby, A. J.; Ligon, K. L.; Wiseman, J. M.; Cooks, R. G.; Agar, N. Y. R. Angew Chem Int Edit 2010, 49, 5953-5956.

What is claimed is:

1. An apparatus for producing samples for mass spectrometry analysis, the apparatus comprising:
a solvent dispenser configured to dispense single droplets of solvent on a sample comprising an analyte;
an electrically conductive conduit configured to transfer the droplets of solvent and the analyte from the sample to a mass spectrometer and that allow a voltage potential to be applied to the droplets; and, optionally,
a heated conduit configured to increase the temperature of the droplets of solvent and the analyte.

2. The apparatus of claim 1, comprising a heated conduit configured to increase the temperature of the droplets of solvent and the analyte.

3. The apparatus of claim 1, wherein the solvent dispenser comprises a miniaturized liquid transfer device.

4. The apparatus of claim 1, wherein the solvent dispenser comprises a piezoelectric actuator.

5. The apparatus of claim 1, wherein each of the droplets of solvent are between 5 and 50 picoliters.

6. The apparatus of claim 1, wherein each of the droplets of solvent are between 10 and 30 picoliters.

7. The apparatus of claim 1, wherein each of the droplets of solvent are approximately 22 picoliters.

8. The apparatus of claim 1, wherein the solvent dispenser is configured to dispense droplets of solvent in a grid pattern.

9. The apparatus of claim 1, wherein further comprising a sample retainer configured to retain the sample.

10. The apparatus of claim 9, further comprising an actuator configured to move the sample retainer in two orthogonal directions.

11. The apparatus of claim 9, further comprising an actuator configured to move the sample retainer in three orthogonal directions.

12. The apparatus of claim 1, wherein the droplets of solvent are spaced apart between 1.0 μm and 1.0 mm in the grid pattern.

13. The apparatus of claim 1, wherein the ionization device comprises a heating element and a voltage source.

14. The apparatus of claim 12, wherein the heating element is configured to be heated to a temperature between 250 and 350 Celsius.

15. The apparatus of claim 12, wherein the heating element is configured to be heated to a temperature of approximately 300 Celsius.

16. The apparatus of claim 1, wherein the electrically conductive conduit is a capillary tube comprising a first end proximal to the solvent dispenser and a second end distal from the solvent dispenser.

17. The apparatus of claim 16, wherein the capillary tube comprises an electrically conductive material.

18. The apparatus of claim 17, wherein the electrically conductive material is a metal coating proximal to the first end of the capillary tube.

19. The apparatus of claim 18, wherein the metal coating is platinum.

20. The apparatus of claim 18, wherein ionization device is configured to apply a voltage differential between the metal coating on the capillary tube and the heated conduit.

21. The apparatus of claim 16 wherein the capillary tube comprises an outer diameter between 300 and 400 micrometers (μm) and inner diameter between 50 and 150 micrometers (μm).

22. The apparatus of claim 16, wherein the capillary tube comprises an outer diameter of approximately 360 micrometers (μm) and inner diameter of approximately 100 micrometers (μm).

23. The apparatus of claim 16, wherein the capillary tube is a silica tube.

24. The apparatus of claim 1, further comprising a mass spectrometer coupled to the conduit.

25. The apparatus of claim 1 wherein the solvent dispenser comprises:
a plurality of pneumatic lines;
a plurality of reservoirs; and
a plurality of dispensing tips, wherein the plurality of pneumatic lines are configured to transport solvent out of the plurality of reservoirs and into dispensing tips.

26. A method for imaging a surface comprising:
(a) applying a discrete volume of a solvent to a plurality of distinct sites on the surface, the discrete volume of solvent being applied through a dispenser;
(b) individually collecting and ionizing the discrete volumes of applied solvent to obtain a plurality of ionized liquid samples, wherein the collecting is through a solvent conduit; and
(c) individually subjecting the plurality of ionized liquid samples to mass spectrometry analysis.

27. The method of claim 26, wherein the plurality of distinct sites are spaced essentially uniformly from one another across the surface.

28. The method of claim 26, wherein the plurality of distinct sites are arranged in a grid patter over the surface.

29. The method of claim 26, wherein the plurality of distinct sites comprise at least 10 sites.

30. The method of claim 29, wherein the plurality of distinct sites comprise 100 to 5,000 sites.

31. The method of claim 26, wherein the location of each of the plurality of distinct sites is recorded and correlated to the mass spectrometry analysis obtained for the liquid sample corresponding to the site.

32. The method of claim 31, further comprising producing an array of data from the mass spectrometry analysis of the plurality of sites to image the surface.

33. The method of claim 26, wherein the plurality of distinct sites are separated by about 1.0 μm to 1.0 mm.

34. The method of claim 26, wherein the method if automated.

35. The method of claim 34, wherein steps (a) and (b) are performed by a robot.

36. The method of claim 26, wherein the discrete volume of a solvent is not applied as a spray.

37. The method of claim 26, wherein the discrete volume of a solvent is applied as a droplet.

38. The method of claim 26, wherein the discrete volume of a solvent is between 5 and 50 or 10 and 30 picoliters.

39. The method of claim 26, wherein the discrete volume of a solvent is applied at using a pressure of less than 100 psig.

40. The method of claim 26, wherein discrete volume of a solvent is applied at using a pressure of less than 10 psig.

41. The method of claim 26, wherein individually collecting and ionizing the discrete volumes comprises applying an electrical potential and/or heat to the collected solvent.

42. The method of claim 26, wherein applying heat comprises heating to a temperature between 250 and 350 Celsius.

43. The method of claim 41, wherein the electrical potential comprises at least 0.5 kV.

44. The method of claim 43, wherein the electrical potential comprises between about 1.0 and 5.0 kV.

45. The method of claim 26, wherein discrete volume of a solvent is applied using a mechanical pump to move the solvent through the dispenser.

46. The method of claim 26, wherein the discrete volume of a solvent is applied using a piezoelectric actuator to move the solvent through the dispenser.

47. The method of claim 26, wherein the solvent conduit is a capillary tube.

48. The method of claim 26, wherein the solvent conduit is composed of silica.

49. The method of claim 26, wherein the solvent conduit has an inner diameter between 50 and 150 micrometers (μm).

50. The method of claim 26, wherein the solvent conduit comprises an electrically conductive material.

51. The method of claim 50, wherein the electrically conductive material is a metal coating.

52. The method of claim 51, wherein the metal coating is platinum.

53. The method of claim 52, wherein the solvent is applied through a dispenser that is separate from the collection conduit.

54. The method of claim 26, wherein the solvent comprises methanol, chloroform, formic acid, water, dimethylformamide (DMF) or acetonitrile (ACN).

55. The method of claim 26, wherein the solvent comprises a mixture of DMF and ACN.

56. The method of claim 26, wherein the solvent is essentially free of water.

57. The method of claim 26, wherein the solvent comprises water.

58. The method of claim 26, wherein the solvent comprises an agent that increases surface tension.

59. The method of claim 26, wherein the solvent comprises a surfactant or a supercharging reagent.

60. The method of claim 26, wherein collecting the applied solvent is between 0.05 and 10 seconds after the applying step.

61. The method of claim 26, wherein the surface comprises a biological material.

62. The method of claim 61, wherein the biological material is a tissue section.

63. The method of claim 61, wherein the biological material is resected tissue from a subject.

64. The method of claim 63, wherein the resected tissue is a tumor.

65. The method of claim 26, wherein the mass spectrometry comprises ambient ionization MS.

66. The method of claim 26, wherein the method is performed using an apparatus in accordance with any one of claims 1-24.

67. The method of claim 26 wherein:
the dispenser comprises a plurality of pneumatic lines, a plurality of reservoirs, and a plurality of dispensing tips; and
applying the discrete volume of solvent through the dispenser comprises transporting the solvent from plurality of reservoirs, through the plurality of pneumatic lines and into dispensing tips.
